SEQUENCE LISTING (142 sequences)

SEQ ID NO: 1 - DNA, 10939 bp, Cricetulus griseus
gagctcaattaaccctcactaaagggagtcgactcgatcctttacagaaaacttgcaaaccctcttggagtagaaaagtagtagtatctgacacaagtatcagcaaaatgcaaacttctccccatccccagaaaaccattataaaaacccccatatcttatgcccaactgtagtgatatattatttatgatttattaaaacttgcttaaggattcagaaagcaaagtcagccttaagctatagagaccaggcagtcagtggtggtacacacctttaatcccaggactcaggattaagaagtagacggacctctgttagttcaagtctaccattacctacacaagagtgaagagtaaccgatctcatgcctttgatcccagcagctgggatcatgtgcattcaatcccagcattcgggagttatataagacaggagcaaggtctcagagctggcattcattctccagccacattgaggataggaaaacattgaagtgtcaggatgctgaggagaggcagcagtttgaggtttggtagaaccaggatcaccttttggtctgaggtagagtaagaactgtggctggctgctttgcttttctgatcttcagcttgaagcttgaactccaatatttgtctctgggtctattattatcatgttacacctaactttaaagctgatttacgcaagacagttgtaggtggacctttctttcctgcccaccagttcccaaataactgacacggagactcaatattaattataaatgattggttaatagctcagtcttgttactggctaactcttacattttaaattaactcatttccatccctttacttgctgccatgtggttcatggcttgttcaagtcctgcttcttctgtctctggctggtgatgcctctggttctgccctttatcccagaattctcctagtctggctctcctgcccagctataggccagtcagctgtttattaaccaatgagaataatacatatttatagtgtacaaagattgctcctcaacacccaattttttatgtgcaacctgagaatctggactcattgccctcatgcttgcagaggcggcacccttacccactaagccacctttctagccctgttgcttttgttttttgagacaggttccactatgtagcccaggctggcctcaaactgaccattctcctgcctaaacctcccgaacactggaattatagtcaaggcctacctgccctggcattttcacacttttatttcctggctgagtccattgactttacactcatcaaggttgaaccagttggagtttaattacagtgccaatcgcactgaatcccacataatcaaacaacttcaaggaagcaaaaaaccagtttttcctgaagatcaatgtcagcttgcctgattcagaatagacccccgaaaaaaggcaaatgcttgataaccaatttcttcttattgttcaatcccctgctgctgtgtgtaagctcctgagaaaggacagtaaggggacattcatgatcagagaaagagccccaactccccccccagccccacccccaccctgtccacagtctgttggtttggtttccccctggctgacacccagaaatcacaacataatcacctaggtcactgtaacaagttcctttctggaaaatgctacaaatgatattggtaacatgagtaatgaataatgcctggagtccaactcccttgtgacccagcaatgttttccgtgggtgctcccttccccagctgcaggcctgacatgtaccttaaaaagcctcccctggaggacagaattttgtgggtactatagtgttctcacaaatacttcccctaatacccttacttagttaccataaataacatgcagcccctggtgaggcacacagggctccaatgtacagcttctcagacactgcaggaaccttcctctcctaatgcagcactggtctcttcaggctggacagcaggaacccataccactccaatcctagtgtggagtagagctgtctacgaaaaccagcagatctatagctaaatgtgtttcaattttatgctttgacaaattgtactgaccccacccccaccccttcccccttgctgtgctgggaattgaacccaggaccttgtgcatgccaggcaagtactctaacactgagctatagccccaatctttcatccaagtctctatgtgtgcccacactcgctttttattttgagacaaaaggttcttattttgagataaggtctcactatgttgccttgacttttttttttttttttttttgaacttttgaccttcctacctcagctgagactacaagtcttttaccatcaggcccggctgatggtaaaataacagtatttgaaatagtttaaacacatcatcttaatggtcaaccacacaatttccgaaatgttgctggctcagtctggggcaaacctgtccgccccaacattggtgctaggaagaaagcacagacaagtagccctcccagctcaggagtaaaagacctggagggggtggcccacttcggtcaagttcacgggatggggaggggtaccctcctccagtagtggtggtatttggcagttcctccaccgacgccctctggaagcacctgcttggacccgcaaagccaggaatgcagcttcctcaagggactcgccagcgagggtaacaggacagaggcgtcccaagagggctggggcggaagggggaagacagggtcggccttagatagggcaaagggccttctggctgtgttcccggggtaaccgccccaccacgcctggagcccgacgtggcgagcgatggggacagcgagcaggaagtcgtactggggagggccgcgtagcagatgcagccgagggcggcgctgccaggtacacccgagggcaccgcgggggtgagcgccaggtccctgaaccagccaggcctccagagccgagtccggcggaccgacggtacgttctggaatgggaagggatccgggacaccgaattgctgcattgaggggctcagaggttctgatgtgggagtccagaaagggttttatctaccggaggtgatgtgacttccggcctctggaagtgctgttggagtctctgggaccttgggtcctctcgactaggtttggaaggggtgaaataggggtagggagaaaggagaggactgcagcaatgtcttcccgaacgacctgggttcgggaggggtcgaaggacaaggggctgttgtggggggtcttcagacgcggaggggtggtattctattttctgggaagatggtgtcgatgcacttgaccaagtctagtcgatctgaagaggctaggggaacagacagtgagagaggatggtggagggagtggcagaacccttccagaaactgggagaggctctagcacctgcaaccccttccctggcctccggggagtcccagaagagggcaggaccatggacacaggtgcattcgtgccggcgcgctccggcctggcgaaggtgcgcgctcttggaggccgcgggagggccagacgcgcgcccggagagctggccctttaaggctacccggaggcgtgtcaggaaatgcgccctgagcccgcccctcccggaacgcggcccgagacctggcaagctgagacggaactcggaactagcactcggctcgcggcctcggtgaggccttgcgcccgccatgcctctgtcattgcccctcgggccgcctccctgaacctccgtgaccgccctgcagtcctccctcccccccttcgactcggcgggcgcttccgggcgctcccgcagcccgccctccacgtagcccacacctccctctcggcgctccgcttcccacgcggtccccgacctgttctttcctcctccaccctgcccttctgtccctctcccttcctttctcccctcgactcgtccctattaggcaacagcccctgtggtccagccggccatggctgtcaaggctcacacccttagctaggccccttctcccttccctgggtcttgtctcatgaccccctgccccgcccgggagcgagcgcgatgtggagcagtgcctctggcaagcagaacttcacccaagccatgtgacaattgaaggctgtacccccagaccctaacatcttggagccctgtagaccagggagtgcttctggccgtggggtgacctagctcttctaccaccatgaacagggcccctctgaagcggtccaggatcctgcgcatggcgctgactggaggctccactgcctctgaggaggcagatgaagacagcaggaacaagccgtttctgctgcgggcgctgcagatcgcgctggtcgtctctctctactgggtcacctccatctccatggtattcctcaacaagtacctgctggacagcccctccctgcagctggatacccctatcttcgtcactttctaccaatgcctggtgacctctctgctgtgcaagggcctcagcactctggccacctgctgccctggcaccgttgacttccccaccctgaacctggaccttaaggtggcccgcagcgtgctgccactgtcggtagtcttcattggcatgataagtttcaataacctctgcctcaagtacgtaggggtggccttctacaacgtggggcgctcgctcaccaccgtgttcaatgtgcttctgtcctacctgctgctcaaacagaccacttccttctatgccctgctcacatgtggcatcatcattggtgagtggggcccgggggctgtgggagcaggatgggcatcgaactgaagccctaaaggtcaacactgtaggtacctttacttactgtcccaggtcccttgcatcagcagttacaggaagagccctgtagaaaacaaataacttccttatggtcattcaacaagttagggacccagccagggtgaaaataatgttagcagcaactacagcaaagatggctctcgccacttgcatgattaaaatgtgccaggtactcagatctaagcattggatccacattaactcaactaatccctattacaaggtaaaatatatccgaattttacagagggaaaaccaaggcacagagaggctaagtagcttgaccaggatcacacagctaataatcactgacatagctgggatttaaacataagcagttacctccatagatcacactatgaccaccatgccactgttccttctcaagagttccaggatcctgtctgtccagttctctttaaagaggacaacacatctgacattgctaccttgaggtaacatttgaaatagtgggtagacatatgttttaagttttattcttactttttatgtgtgtgtgtttggggggccaccacagtgtatgggtggagataaggggacaacttaagaattggtcctttctcccaccacatgggtgctgaggtctgaactcaggtcatcaggattggcacaaatccctttacccactgagccatttcactggtccaatatatgtgtgcttttaagaggctttaactattttcccagatgtgaatgtcctgctgatcattatccccttttacccggaagccctctgggaggtgccatccctgtggtcgtctgcatacaaatggggaaactgcaactcagagaaacaaggctacttgccagggccccacaagtaagataggctgggatgccatcccagactggccacactccctggcctgtgcttcaagccagtttactttgttcctgcccattggaagttagcatgttgcagtcaaacacaataactacaggccaaaagtgcttttaaattaaagtcagatgaacttttaaacatccagagctcctcaactgcaggagttacaacctgattctgcaaccatctttgcagtgcccggtagtcatatgtagctagaggctcttggctaggacagcatgtgttaggaaacatctggccctgagatcattgaattgagtgactgctgggtgacaaagaccaaggcatccgttccctgagagtcctgggcaagcagcaatgtgaccttcatttgtacctactcaggttctttatctgtcctgtttgacctacttagtctcctctggtgtctcagaggcccaggctgggtactctggatgtcaggatcaggccaatgcgcacatctgccctagaaatgtccccctggttgagcagctcctgaatccatcggtaaagggtctggaccagggaggagtcagataaaaagctgacagcactgggggactccatggggaactcccacctgcccccacacatccatcctaagagaactggtattccttgtttcctctttgtcctacaaggcaccctgggatcccacttcagtctcccagccttgccagggttagagggcatgagcctccttgtggggaatttagatgcaagaaggtacagtcactagagaacctgagctcagatccccaaagtaaccagtacctgatagtgaggcagctgagaaccgcagcagcctgcctgagtggctgaactctgcggcctccggaactggccccaactgttgggtctcctcttccttcctcctgtgagggagggcccatctctgataagtgctgtggggactctagagtagggaggaggaggagcaatctaagcaggccttactgagaagtccttgctggcatgtggctgcctgaggagtacagactgggaacacccatttgaatgagtaaggtttttcctgaaggccatggggagccacggaggaaaatcattttagttacaagacaaagagtagattggttaacatgggagcaaggacatggccccaattttcatagatgaaggaaattggaactcagagaggttaagtaacttctcccaaatagctcagcttcaaaatcacagaacagtcagagtctagatctctctgatgcctgtgatggtcctgccattccatgttgctgatccctgtggcatcagtaagcctctaccttgtgggaatgcaggatctaaatgaagagaggaagtgctggccccatgctgtggtctggaaagctatgcaggctctttgagcagagagtgacccacaagtgaatagagtcctatgagactcaaagcaacatccacccttaagcagctctaaccaaatgctcacactgagggagccaaagccaagttagagtcctgtgcttgcccaaggtcactttgcctggccctcctcctatagcacccgtgttatcttatagccctcattacagtgattacaattataattagagaggtaacagggccacactgtccttacacattcccctgctagattgtagctgggagagggggagatgtaggtggctgggggagtgggagggaagatgcagattttcattctgggctctactccctcagccattttttggtgtgggagttagactttggatatgttgatgatgaggtaagggccacagaacagtctgaactgtggtatcagaatcctgtccctctccctctctcctcatccctcttcaccttgtcactcctctgtctgctacaggtggtttctggctgggtatagaccaagagggagctgagggcaccctgtccctcataggcaccatcttcggggtgctggccagcctctgcgtctccctcaatgccatctataccaagaaggtgctcccagcagtggacaacagcatctggcgcctaaccttctataacaatgtcaatgcctgtgtgctcttcttgcccctgatggttctgctgggtgagctccgtgccctccttgactttgctcatctgtacagtgcccacttctggctcatgatgacgctgggtggcctcttcggctttgccattggctatgtgacaggactgcagatcaaattcaccagtcccctgacccacaatgtatcaggcacagccaaggcctgtgcgcagacagtgctggccgtgctctactatgaagagactaagagcttcctgtggtggacaagcaacctgatggtgctgggtggctcctcagcctatacctgggtcaggggctgggagatgcagaagacccaagaggaccccagctccaaagagggtgagaagagtgctattggggtgtgagcttcttcagggacctgggactgaacccaagtggggcctacacagcactgaaggcttcccatggagctagccagtgtggccctgagcaatactgtttacatcctccttggaatatgatctaagaggagccagggtctttcctggtaatgtcagaaagctgccaaatctcctgtctgccccatcttgttttgggaaaaccctaccaggaatggcacccctacctgcctcctcctagagcctgtctacctccatatcatctctggggttgggaccagctgcagccttaaggggctggattgatgaagtgatgtcttctacacaagggagatgggttgtgatcccactaattgaagggatttgggtgaccccacacctctgggatccagggcaggtagagtagtagcttaggtgctattaacatcaggaacacctcagcctgcctttgaagggaagtgggagcttggccaagggaggaaatggccattctgccctcttcagtgtggatgagtatggcagacctgttcatggcagctgcaccctggggtggctgataagaaaacattcacctctgcatttcatatttgcagctctagaacgggggagagccacacatcttttacgggttaagtagggtgatgagctcctccgcagtccctaaccccagctttacctgcctggcttcccttggcccagctacctagctgtactccctttctgtactcttctcttctccgtcatggcctcccccaacacctccatctgcaggcaggaagtggagtccacttgtaacctctgttcccatgacagagccctttgaatacctgaacccctcatgacagtaagagacatttatgttctctggggctggggctgaaggagcccactggttctcacttagcctatctggctcctgtcacaaaaaaaaaaaaagaaaaaaaaaaagcataaaccaagttactaagaacagaagttggtttataacgttctggggcagcaaagcccagatgaagggacccatcgaccctctctgtccatatcctcatgctgcagaagtacaggcaagctcctttaagcctcatataggaacactagcctcactcatgagggttttactccatgacctgtcaacctcaaagccttcaacatgaggactccaacgtaaatttggggacagaagcactcagaccataccccagcaccacaccctcctaacctcagggtagctgtcattctcctagtctcctctcttgggcctttagaacccccatttccttggggtaatgtctgatgtttttgtccctgtcataaaaagatggagagactgtgtccagcctttgattcctacttcctacaatcccaggttctaatgaagtttgtggggcctgatgccctgagttgtatgtgatttaataataaaaaagcaagatacagcatgtgtgtggactgagtgagggccacagggatctaaaagccaagtgtgaggggacccagctacagcaggcagcatcctgagcctggaatctcttcaggacaagaattctccatatacctacctactctggggagtaggtggccagagttcaagcttcccttagtaccaactaccactggctgtgctcttactgaaggcagacatggcactgagtgctgtccatctgtcactcatctccacagccattcctaatgtgtggggtgggagccatcaccaaaccccattttcagataaggacacaggctcagagaggcttgtgtggagaaaagtagcagcagaattcagagagctgggtctcctgcagcaccttggactgccagcagccacagtgcttgtcacacagcacatactcaaaagaatgccagccccctcagcctagagtgcctggcctttctttcagatgaggaagagggtcaaagctgttagcttgcccaccatatgaccacatacatgaccaacagcttgagggagggaggattactgtggctcccagcctgagaggtgggacacccaaatgtattaggtccttgaatcagggctgaccttgtgattcagtcactcctaccagaatgctggggaatggggatgccaaaggcaaaggaggctttctaaggtgtggtgtaagataggcatttctgcttccatgtacacctgtgagcagagtaggaaggccctgtggagaatatatcccacaaaccagtagcccttcctggcagtgggtgaatactgccaccctatacccctatgcaaggccagtagaaccacccaacccacaacatctagagaaattacaggtcatcttaagcctctaaattgtggagaaactcgacatgcgcacgattcctaacctgctagcctagggtgcggggtggataatttaaggaaactggggtttcttatagaatcggaggctccatgaagtcaccctgacaagaggtcagcaatagccagcagcagtggctactcctaagcctccagacagagcaccctgtgaatgtaccttattctcacatctgggtgtctataggtgtgactgggtcagatgtcacccaggccattgcaatgggcccttagccccatggggtgttgggatagcagccaagcagctcccatgctgagatactgcctgcagtagactgatggataagaaaacaaggcccaaaatgttttctttccagacttgatctttctttgttcaaaaatgctgttttcccttaaacttgcccaaacccattgttttgcagttgaggaaaataaggcatagaaagattaaaggaagtttctgaggttacagagcaaagtactggcttcacctgaaatagacaggtgtgccctgatcctgatttgagctc

SEQ ID NO: 2 - protein, 352 aa, Cricetulus griseus
MALTGGSTASEEADEDSRNKPFLLRALQIALVVSLYWVTSISMVFLNKYLLDSPSLQLDTPIFVTFYQCLVTSLLCKGLSTLATCCPGTVDFPTLNLDLKVARSVLPLSVVFIGMISFNNLCLKYVGVAFYNVGRSLTTVFNVLLSYLLLKQTTSFYALLTCGIIIGGFWLGIDQEGAEGTLSLIGTIFGVLASLCVSLNAIYTKKVLPAVDNSIWRLTFYNNVNACVLFLPLMVLLGELRALLDFAHLYSAHFWLMMTLGGLFGFAIGYVTGLQIKFTSPLTHNVSGTAKACAQTVLAVLYYEETKSFLWWTSNLMVLGGSSAYTWVRGWEMQKTQEDPSSKEGEKSAIGV

SEQ ID NO: 3 - DNA, 32 bp, Artificial Sequence
  Synthetic Primer
ggatcctgcgcatgaaaaagcctgaactcacc

SEQ ID NO: 4 - DNA, 29 bp, Artificial Sequence
  Synthetic Primer
gcggccgcctattcctttgccctcggacg

SEQ ID NO: 5 - DNA, 33 bp, Artificial Sequence
  Synthetic Primer
atgcatgccaccatgaaaaagcctgaactcacc

SEQ ID NO: 6 - DNA, 28 bp, Artificial Sequence
  Synthetic Primer
ggatcccaggctttacactttatgcttc

SEQ ID NO: 7 - DNA, 25 bp, Artificial Sequence
  Synthetic Primer
gctgtctggagtactgtgcatctgc

SEQ ID NO: 8 - DNA, 27 bp, Artificial Sequence
  Synthetic Primer
ggaatgcagcttcctcaagggactcgc

SEQ ID NO: 9 - DNA, 27 bp, Artificial Sequence
  Synthetic Primer
tgcatcaggtcggagacgctgtcgaac

SEQ ID NO: 10 - DNA, 27 bp, Artificial Sequence
  Synthetic Primer
gcactcgtccgagggcaaaggaatagc

SEQ ID NO: 11 - DNA, 24 bp, Artificial Sequence
  Synthetic Primer
tgtgctgggaattgaacccaggac

SEQ ID NO: 12 - DNA, 22 bp, Artificial Sequence
  Synthetic Primer
ctacttgtctgtgctttcttcc

SEQ ID NO: 13 - DNA, 27 bp, Artificial Sequence
  Synthetic Primer
ctcgactcgtccctattaggcaacagc

SEQ ID NO: 14 - DNA, 27 bp, Artificial Sequence
  Synthetic Primer
tcagaggcagtggagcctccagtcagc

SEQ ID NO: 15 - DNA, 31 bp, Artificial Sequence
  Synthetic Primer
gatatcatggccgggaccgtgcgcaccgcgt

SEQ ID NO: 16 - DNA, 31 bp, Artificial Sequence
  Synthetic Primer
gctagctcagtgcaccaggaagaagaagcac

SEQ ID NO: 17 - DNA, 1743 bp, Artificial Sequence
  Synthetic Primer
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactga

SEQ ID NO: 18 - protein, 580 aa, Artificial Sequence
  Synthetic Primer
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 19 - DNA, 31 bp, Artificial Sequence
  Synthetic Primer
atagaattccaccatggccgggaccgtgcgc

SEQ ID NO: 20 - DNA, 31 bp, Artificial Sequence
  Synthetic Primer
ataggatcccttcagcggggaatgaacgttc

SEQ ID NO: 21 - DNA, 21 bp, Artificial Sequence
  Synthetic Primer
gggccagtggatagacagatg

SEQ ID NO: 22 - DNA, 24 bp, Artificial Sequence
  Synthetic Primer
caggggccagtggatagaccgatg

SEQ ID NO: 23 - DNA, 24 bp, Artificial Sequence
  Synthetic Primer
caggggccagtggatagactgatg

SEQ ID NO: 24 - DNA, 23 bp, Artificial Sequence
  Synthetic Primer
gctcactggatggtgggaagatg

SEQ ID NO: 25 - DNA, 1392 bp, Mus musculus
atgaacttcgggctcaccttgattttccttgtccttactttaaaaggtgtccagtgtgaggtgcaactggtggagtctgggggaggcttagtgaagcctggaggatccctgaaactctcctgtgcagcctctggattcactttcagtcgctatgccatgtcttgggttcgccagattccagagaagatactggagtgggtcgcagccattgatagtagtggtggtgacacctactatttagacactgtgaaggaccgattcaccatctccagagacaatgccaataataccctgcacctgcaaatgcgcagtctgaggtctgaggacacagccttgtattactgtgtaagacaggggggggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - DNA, 342 bp, Mus musculus
gaggtgcacctggtggagtctgggggaggcttagtgaagcctggagggtccctgaaactctcctgtgcagcctctggattcactttcagtaactatgccatgtcttgggttcgccagactccagagaagaggctggagtgggtcgcagccattaataataatggtgatgacacctactatttagacactgtgaaggaccgattcaccatctccagagacaatgccaagaacaccctgtacctgcaaatgagcagtctgaggtctgaggacacagccctgtattactgtgtaagacaagggggggcttactggggccaagggactctggtcactgtctctgca

SEQ ID NO: 27 - DNA, 1413 bp, Mus musculus
atgggatggaactggatctttattttaatcctgtcagtaactacaggtgtccactctgaggtccagctgcagcagtctggacctgagctggtgaagcctggggcttcagtgaagatatcctgcaaggcttctggttactcattcactggctactacatgcactgggtgaagcaaagtcctgaaaagagccttgagtggattggagagattaatcctagcactggtggtactacctacaaccagaagttcaaggccaaggccacattgactgtagacaaatcctccagcacagcctacatgcagctcaagagcctgacatctgaggactctgcagtctattactgtgcaaggaggggcggattaactgggacgagcttctttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - DNA, 354 bp, Mus musculus
caggtcactctgaaagagtctggccctgggatattgcagccctcccagaccctcagtctgacttgttctttctctgggttttcactgagcacttatggtatgggtgtaggttggattcgtcagccttcagggatgggtctggagtggctggccaacatttggtggtatgatgctaagtactataactctgacctgaagagccggctcacaatctccaaggatacctccaacaaccaggtgttcctcaagatctccagtgtggacacttcagatactgccacatactactgtgctcaaatgggactggcctggtttgcttactggggccaagggactctggtcactgtctctgca

SEQ ID NO: 29 - DNA, 354 bp, Mus musculus
caggtcactctgaaagagtctggccctgggatattgcagccctcccagaccctcagtctgacttgttctttctctgggttttcactgagcatttatggtatgggtgtaggttggattcgtcagccttcagggaagggtctggagtggctggccaacatttggtggaatgatgataagtactataactcagccctgaagagccggctcacaatctccaaggatacctccaacaaccaggtattcctcaagatctccagtgtggacactgcagatactgccacatactactgtgctcaaataggttacttctactttgactactggggccaaggcaccactctcacagtctcctca

SEQ ID NO: 30 - DNA, 1416 bp, Mus musculus
atgaacttcgggctcaccttgattttcctcgtccttactttaaaaggtgtccagtgtgaggtgcagctggtggagtctgggggagacttagtgaagcctggagggaccctgaaactctcctgtgcagcctctggatccactttcagtaactatgccatgtcttgggttcgccagactccagagaagaggctggagtgggtcgcagccattgatagtaatggaggtaccacctactatccagacactatgaaggaccgattcaccatttccagagacaatgccaagaacaccctgtacctgcaaatgaacagtctgaggtctgaagacacagccttttatcactgtacaagacataatggagggtatgaaaactacggctggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - DNA, 366 bp, Mus musculus
gaggtgcagctggtggagtctgggggagacttagtgaagcctggagggtccctgaaactctcctgtgcagcctctggattcactttcagtagctatgccatgtcttgggttcgccagactccagagaagaggctggagtgggtcgcagccattaatagtaatggaggtaccacctactatccagacactatgaaggaccgattcaccatctccagagacaatgccaagaacaccctgtacctgcaaatgagcagtctgaggtctgaagactcagccttgtattactgtacaagacataatggagggtatgaaaactacggctggtttgcttactggggccaagggactctggtcactgtctctgca

SEQ ID NO: 32 - DNA, 1413 bp, Mus musculus
atggaatctaactggatacttccttttattctgtcggtagcttcaggggtctactcagaggttcagctccagcagtctgggactgtgctggcaaggcctggggcttcagtgaagatgtcctgcaaggcttctggctacacctttactggctactggatgcgctgggtaaaacagaggcctggacagggtctggaatggattggcgctatttatcctggaaatagtgatacaacatacaaccagaagttcaagggcaaggccaaactgactgcagtcacatctgtcagcactgcctacatggaactcagcagcctgacaaatgaggactctgcggtctattactgttcaagatcgggggacctaactggggggtttgcttactggggccaagggactctggtcactgtctctacagccaaagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 33 - DNA, 357 bp, Mus musculus
gaggttcagctccagcagtctgggactgtgctggcaaggcctggggcttcagtgaagatgtcctgcaaggcttctggctacacctttaccggctactggatgcactgggtaaaacagaggcctggacagggtctggaatggattggcgctatttatcctggaaatagtgatactaactacaaccagaagttcaagggcaaggccaaactgactgcagtcacatctgccagcactgcctacatggagctcagcagcctgacaaatgaggacgctgcggtctatcactgtacaagatcgggggacctaactggggggcttgcttactggggccaagggactctggtcactgtctctgca

SEQ ID NO: 34 - DNA, 372 bp, Mus musculus
caggtccagctgcagcagcctggggctgaactggtgaagcctggggcttcagtgaaactgtcctgcaaggcttctggatacaccttcactagctactggatgcattgggtgaagcagaggcctggacaaggccttgagtggatcggagagattgatccttctgatagttatacttactacaatcaaaagttcaggggcaaggccacattgactgtagacaaatcctccaacacagcctacatgcaactcagcagcctgacatctgaggactctgcggtctattactgttcaagatcaaatctgggtgatggtcactaccggtttcctgcgtttccttactggggccaagggactctggtcactgtctctgca

SEQ ID NO: 35 - DNA, 372 bp, Mus musculus
caggtccaactgcagcagcctggggctgaactggtgaaacctggggcttcagtgaagctgtcctgcaaggcttctggctacaccttcaccagctactggatgcactgggtgaaacagaggcctggacaaggccttgaatggattggtacaattgacccttctgatagtgaaactcactacaatctacagttcaaggacacggccacattgactgtagacaaatcctccagcacagcctacatgcagctcagcagcctgacatctgaggactctgcggtctattattgtataagaggcgccttctatagttcctatagttactgggcctggtttgcttactggggccaagggactctggtcactgtctctgca

SEQ ID NO: 36 - protein, 463 aa, Mus musculus
MNFGLTLIFLVLTLKGVQCEVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 114 aa, Mus musculus
EVHLVESGGGLVKPGGSLKLSCAASGFTFSNYAMSWVRQTPEKRLEWVAAINNNGDDTYYLDTVKDRFTISRDNAKNTLYLQMSSLRSEDTALYYCVRQGGAYWGQGTLVTVSA

SEQ ID NO: 38 - protein, 470 aa, Mus musculus
MGWNWIFILILSVTTGVHSEVQLQQSGPELVKPGASVKISCKASGYSFTGYYMHWVKQSPEKSLEWIGEINPSTGGTTYNQKFKAKATLTVDKSSSTAYMQLKSLTSEDSAVYYCARRGGLTGTSFFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 118 aa, Mus musculus
QVTLKESGPGILQPSQTLSLTCSFSGFSLSTYGMGVGWIRQPSGMGLEWLANIWWYDAKYYNSDLKSRLTISKDTSNNQVFLKISSVDTSDTATYYCAQMGLAWFAYWGQGTLVTVSA

SEQ ID NO: 40 - protein, 118 aa, Mus musculus
QVTLKESGPGILQPSQTLSLTCSFSGFSLSIYGMGVGWIRQPSGKGLEWLANIWWNDDKYYNSALKSRLTISKDTSNNQVFLKISSVDTADTATYYCAQIGYFYFDYWGQGTTLTVSS

SEQ ID NO: 41 - protein, 471 aa, Mus musculus
MNFGLTLIFLVLTLKGVQCEVQLVESGGDLVKPGGTLKLSCAASGSTFSNYAMSWVRQTPEKRLEWVAAIDSNGGTTYYPDTMKDRFTISRDNAKNTLYLQMNSLRSEDTAFYHCTRHNGGYENYGWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 122 aa, Mus musculus
EVQLVESGGDLVKPGGSLKLSCAASGFTFSSYAMSWVRQTPEKRLEWVAAINSNGGTTYYPDTMKDRFTISRDNAKNTLYLQMSSLRSEDSALYYCTRHNGGYENYGWFAYWGQGTLVTVSA

SEQ ID NO: 43 - protein, 470 aa, Mus musculus
MESNWILPFILSVASGVYSEVQLQQSGTVLARPGASVKMSCKASGYTFTGYWMRWVKQRPGQGLEWIGAIYPGNSDTTYNQKFKGKAKLTAVTSVSTAYMELSSLTNEDSAVYYCSRSGDLTGGFAYWGQGTLVTVSTAKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 119 aa, Mus musculus
EVQLQQSGTVLARPGASVKMSCKASGYTFTGYWMHWVKQRPGQGLEWIGAIYPGNSDTNYNQKFKGKAKLTAVTSASTAYMELSSLTNEDAAVYHCTRSGDLTGGLAYWGQGTLVTVSA

SEQ ID NO: 45 - protein, 124 aa, Mus musculus
QVQLQQPGAELVKPGASVKLSCKASGYTFTSYWMHWVKQRPGQGLEWIGEIDPSDSYTYYNQKFRGKATLTVDKSSNTAYMQLSSLTSEDSAVYYCSRSNLGDGHYRFPAFPYWGQGTLVTVSA

SEQ ID NO: 46 - protein, 124 aa, Mus musculus
QVQLQQPGAELVKPGASVKLSCKASGYTFTSYWMHWVKQRPGQGLEWIGTIDPSDSETHYNLQFKDTATLTVDKSSSTAYMQLSSLTSEDSAVYYCIRGAFYSSYSYWAWFAYWGQGTLVTVSA

SEQ ID NO: 47 - DNA, 717 bp, Mus musculus
atgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaattggactctggagcccctgacaggttcactggcagtggatcagggacagatttcacactgaaaatcagtagagtggaggctgaggatttgggaatttattattgctggcaaggtacacattttccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 48 - DNA, 336 bp, Mus musculus
gatgttgtgatgacccagtctccactcactttgtcgattaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttctcactgaaaatcagcagagtggaggctgaggatttgggaatttattattgctggcaaggtacacattttccgctcacgttcggtgctgggaccaagctggagctgaaa

SEQ ID NO: 49 - DNA, 717 bp, Mus musculus
atgagtcctgtccagttcctgtttctgttaatgctctggattcaggaaaccaacggtgatgttgtgatgacccagactccactgtctttgtcggttaccattggacaaccagcctctatctcttgcaagtcaagtcagagcctcttatatagtaatggaaagacatatttgaattggttacaacagaggcctggccaggctccaaagcacctaatgtatcaggtgtccaaactggaccctggcatccctgacaggttcagtggcagtggatcagaaacagattttacacttaaaatcagcagagtggaggctgaagatttgggagtttattactgcttgcaaagtacatattatccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 50 - DNA, 324 bp, Mus musculus
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaattaattattgtctacagtgtgatgagtttcctccgtggacgttcggtggaggcaccaagctggaaatcaaa

SEQ ID NO: 51 - DNA, 336 bp, Mus musculus
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 52 - DNA, 705 bp, Mus musculus
atgagaccctccattcagttcctggggctcttgttgttctggcttcatggtgttcagtgtgacatccagatgacacagtctccatcctcactgtctgcatctctgggaggcaaagtcaccatcacttgcaaggcaagtcaggacattaacaagaatatagtttggtaccaacacaagcctggaaaaggtcctaggctgctcatatggtacacatctacattacagccaggcatcccatcaaggttcagtggaagtgggtctgggagagattattccttcagcatcagcaacctggagcctgaagatattgcaacttattactgtctacagtatgataatcttccacggacgttcggtggaggcaccaaactggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 53 - DNA, 321 bp, Mus musculus
gacatccagatgacacagtctccatcctcactgtctgcatctctgggaggcaaagtcaccatcacttgcaaggcaagtcaggacattaacaagaatataatttggtaccaacacaagcctggaaaaggtcctaggctgctcatatggtacacatctacattacagccaggcatcccatcaaggttcagtggaagtgggtctgggagagattattccttcagcatcagcaacctggagcctgaagatattgcaacttattactgtctacagtatgataatcttccacggacgttcggtggaggcaccaagctggaaatcaaa

SEQ ID NO: 54 - DNA, 720 bp, Mus musculus
atgaggttctctgctcagcttctggggctgcttgtgctctggatccctggatccactgcagatattgtgatgacgcaggctgcattctccaatccagtcactcttggaacatcaacttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtatacgttcggatcggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 55 - DNA, 336 bp, Mus musculus
gatattgtgatgacgcaggctgcattctccaatccagtcactcttggaacatcagcttccatctcctgcaggtctagtaagagtctcctacatagtaatggcatcacttatttgtattggtttctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtatacgttcggatcggggaccaagctggaaataaaa

SEQ ID NO: 56 - DNA, 321 bp, Mus musculus
gatattgtgctaactcagtctccagccaccctgtctgtgactccaggagacagagtcagtctttcctgcagggccagccatagtattagcaacttcctacactggtatccacaaaaatcacatgagtctccaaggcttctcatcaagtatgcttcccagtccatctctgggatcccctccaggttcagtggcaatggatcagggacagatttcactctcagtatcaacagtgtggagactgaagattttggaatgtatttctgtcaacagagtaacatctggtcgctcacgttcggtgctgggaccaagctggagctgaaa

SEQ ID NO: 57 - DNA, 333 bp, Mus musculus
gacattgtgctcacccaatctccaacttctttggctgtgtctctagggcagagtgtcaccatctcctgcagagccagtgaaagtgttgaatattatggcactagtttaatgcagtggtaccaacagaaaccaggacagccacccaaactcctcatctatggtgcatccaacgtagaatctggggtccctgccaggtttagtggcagtgggtctgggacagacttcagcctcaacatccatcctgtggaggaggatgatattgcaatgtatttctgtcagcaaagtaggaaggttccgtatacgttcggatcggggaccaagctggaaataaaa

SEQ ID NO: 58 - protein, 238 aa, Mus musculus
MSPAQFLFLLVLWIRETNGDVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 112 aa, Mus musculus
DVVMTQSPLTLSITIGQPASISCKSSQSLLDSDGKTYLNWLLQRPGQSPKRLIYLVSKLDSGVPDRFTGSGSGTDFSLKISRVEAEDLGIYYCWQGTHFPLTFGAGTKLELK

SEQ ID NO: 60 - protein, 238 aa, Mus musculus
MSPVQFLFLLMLWIQETNGDVVMTQTPLSLSVTIGQPASISCKSSQSLLYSNGKTYLNWLQQRPGQAPKHLMYQVSKLDPGIPDRFSGSGSETDFTLKISRVEAEDLGVYYCLQSTYYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 108 aa, Mus musculus
DIKMTQSPSSMYASLGERVTITCKASQDINNYLSWFQQKPGKSPKTLIYRANRLVDGVPSRFSGSGSGQDYSLTISSLEYEDMGINYCLQCDEFPPWTFGGGTKLEIK

SEQ ID NO: 62 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 63 - protein, 234 aa, Mus musculus
MRPSIQFLGLLLFWLHGVQCDIQMTQSPSSLSASLGGKVTITCKASQDINKNIVWYQHKPGKGPRLLIWYTSTLQPGIPSRFSGSGSGRDYSFSISNLEPEDIATYYCLQYDNLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 107 aa, Mus musculus
DIQMTQSPSSLSASLGGKVTITCKASQDINKNIIWYQHKPGKGPRLLIWYTSTLQPGIPSRFSGSGSGRDYSFSISNLEPEDIATYYCLQYDNLPRTFGGGTKLEIK

SEQ ID NO: 65 - protein, 239 aa, Mus musculus
MRFSAQLLGLLVLWIPGSTADIVMTQAAFSNPVTLGTSTSISCRSSKSLLHSNGITYLYWYLQKPGQSPQLLIYQMSNLASGVPDRFSSSGSGTDFTLRISRVEAEDVGVYYCAQNLELPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 112 aa, Mus musculus
DIVMTQAAFSNPVTLGTSASISCRSSKSLLHSNGITYLYWFLQKPGQSPQLLIYQMSNLASGVPDRFSSSGSGTDFTLRISRVEAEDVGVYYCAQNLELPYTFGSGTKLEIK

SEQ ID NO: 67 - protein, 107 aa, Mus musculus
DIVLTQSPATLSVTPGDRVSLSCRASHSISNFLHWYPQKSHESPRLLIKYASQSISGIPSRFSGNGSGTDFTLSINSVETEDFGMYFCQQSNIWSLTFGAGTKLELK

SEQ ID NO: 68 - protein, 111 aa, Mus musculus
DIVLTQSPTSLAVSLGQSVTISCRASESVEYYGTSLMQWYQQKPGQPPKLLIYGASNVESGVPARFSGSGSGTDFSLNIHPVEEDDIAMYFCQQSRKVPYTFGSGTKLEIK

SEQ ID NO: 69 - DNA, 333 bp, Mus musculus
cagatccagttggagcagtctggacctgagctgaagaagcctggagagacagtcaagatctcctgcaaggcttctggttatattttcagagactattcaatgcactgggtgaagcaggctccaggaaagggtttaaagtggatgggctggataaacactgagacgggtgagccaacatatgcagatgacttcaagggacggtttgccttctctttggaaacctctgccagcactgcctatttgcagatcaacaacctcaaaaatgaggacacggctacatatttctgtactagcctttactggggccaagggactctggtcactgtctctgca

SEQ ID NO: 70 - DNA, 372 bp, Mus musculus
caggtcactctgaaagagtctggccctgggatattgcagccctcccagaccctcagtctgacttgttctttctctgggttttcactgagcacttatggtatgggtgtaggttggattcgtcagccttcagggaagggtctggagtggctggccaacatttggtggcatgatgataagtactataactcagccctgaagagccggctcacaatctccaaggatatctccaacaaccaggtattcctcaagatctccagtgtggacactgcagatactgccacatactactgtgctcaaatagcccctcgatataataagtacgaaggcttttttgctttctggggccaagggactctggtcactgtctctgca

SEQ ID NO: 71 - DNA, 345 bp, Mus musculus
caggttcaactgcagcagtctggggctgagctggtgaggcctggggcttcagtgaagctgtcctgcaaggcttcgggctacacatttactgactatgaaatgcactgggtgaagcagacacctgtgcatggcctaaaatggattggagctcttgatcctaaaactggtgatactgcctacagtcagaagttcaagggcaaggccacactgactgcagacaaatcctccagcacagcctacatggagctccgcagcctgacatctgaggactctgccgtctattactgtacaagattctactcctatacttactggggccaagggactctggtcactgtctctgca

SEQ ID NO: 72 - DNA, 357 bp, Mus musculus
gaggtgcagcttgttgagactggtggaggactggtgcagcctgaagggtcattgaaactctcatgtgcagcttctggattcagcttcaatatcaatgccatgaactgggtccgccaggctccaggaaagggtttggaatgggttgctcgcataagaagtgaaagtaataattatgcaacatattatggcgattcagtgaaagacaggttcaccatctccagagatgattcacaaaacatgctctatctacaaatgaacaacttgaaaactgaggacacagccatatattactgtgtgagagaggtaactacatcgtttgcttattggggccaagggactctggtcactgtctctgca

SEQ ID NO: 73 - DNA, 369 bp, Mus musculus
gaggtgcagcttgttgagactggtggaggattggtgcagcctaaagggtcattgaaactctcatgtgcagcctctggattcaccttcaatgccagtgccatgaactgggtccgccaggctccaggaaagggtttggaatgggttgctcgcataagaagtaaaagtaataattatgcaatatattatgccgattcagtgaaagacaggttcaccatctccagagatgattcacaaagcatgctctatctgcaaatgaacaacttgaaaactgaggacacagccatgtattactgtgtgagagatccgggctactatggtaacccctggtttgcttactggggccaagggactctggtcactgtctctgca

SEQ ID NO: 74 - protein, 111 aa, Mus musculus
QIQLEQSGPELKKPGETVKISCKASGYIFRDYSMHWVKQAPGKGLKWMGWINTETGEPTYADDFKGRFAFSLETSASTAYLQINNLKNEDTATYFCTSLYWGQGTLVTVSA

SEQ ID NO: 75 - protein, 124 aa, Mus musculus
QVTLKESGPGILQPSQTLSLTCSFSGFSLSTYGMGVGWIRQPSGKGLEWLANIWWHDDKYYNSALKSRLTISKDISNNQVFLKISSVDTADTATYYCAQIAPRYNKYEGFFAFWGQGTLVTVSA

SEQ ID NO: 76 - protein, 115 aa, Mus musculus
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 77 - protein, 119 aa, Mus musculus
EVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSA

SEQ ID NO: 78 - protein, 123 aa, Mus musculus
EVQLVETGGGLVQPKGSLKLSCAASGFTFNASAMNWVRQAPGKGLEWVARIRSKSNNYAIYYADSVKDRFTISRDDSQSMLYLQMNNLKTEDTAMYYCVRDPGYYGNPWFAYWGQGTLVTVSA

SEQ ID NO: 79 - DNA, 336 bp, Mus musculus
gatgttgtgatgacccagactccactcactttgtcggttacccttggacaaccagcctccatctcttgcaagtcaagtcagagcctcttacatagtgatggaaagacatttttgaattggttattacagaggccaggccagtctccaaagcgcctaatctatctggtgtctagactggactctggagtccctgacaggttcactggcagtggatcagggacagatttcacactgaaaatcagcagagtggaggctgaggatttgggagtttattattgctgccaaggtacacattttcctcggacgttcggtggaggcaccaggctggaaatcaaa

SEQ ID NO: 80 - DNA, 336 bp, Mus musculus
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 81 - DNA, 336 bp, Mus musculus
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaaatacacatgttcctcctacgttcggatcggggaccaagctggaaataaaa

SEQ ID NO: 82 - DNA, 336 bp, Mus musculus
gatattgtgatgactcagtctgcaccctctgtacctgtcactcctggagagtcagtatccatctcctgcaagtctagtaagagtctcctgcatagtaatggcaacacttacttgaattggttcctgcagaggccaggccagtctcctcaactcctgatttattggatgtccaaccttgcctcaggagtcccagacaggttcagtggcagtgggtcaggaactgctttcacactgagaatcagtagagtggaggctgaggatgtgggtgtttattactgtatgcaacatatagaataccctttcacgttcggcacggggacaaaattggaaataaaa

SEQ ID NO: 83 - DNA, 336 bp, Mus musculus
gatattgtgatgacgcaggctgcattctccaatccagtcactcttggaacatcagcttccatctcctgcaggtctagtaagagtctcctacatagttatgacatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctccgacgttcggtggaggcaccaagctggaaatcaaa

SEQ ID NO: 84 - DNA, 318 bp, Mus musculus
caaattgttctcacccagtctccagcaatcatgtctgcatttccaggggagaaggtcaccatgacctgcagtgccagctcaagtgttagttacatgtactggtaccagcagaagtcaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtggtgggaccgagctggagctgaaa

SEQ ID NO: 85 - protein, 112 aa, Mus musculus
DVVMTQTPLTLSVTLGQPASISCKSSQSLLHSDGKTFLNWLLQRPGQSPKRLIYLVSRLDSGVPDRFTGSGSGTDFTLKISRVEAEDLGVYYCCQGTHFPRTFGGGTRLEIK

SEQ ID NO: 86 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 87 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 88 - protein, 112 aa, Mus musculus
DIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIK

SEQ ID NO: 89 - protein, 112 aa, Mus musculus
DIVMTQAAFSNPVTLGTSASISCRSSKSLLHSYDITYLYWYLQKPGQSPQLLIYQMSNLASGVPDRFSSSGSGTDFTLRISRVEAEDVGVYYCAQNLELPPTFGGGTKLEIK

SEQ ID NO: 90 - protein, 106 aa, Mus musculus
QIVLTQSPAIMSAFPGEKVTMTCSASSSVSYMYWYQQKSGSSPRLLIYDTSNLASGVPVRFSGSGSGTSYSLTISRMEAEDAATYYCQQWSSYPLTFGGGTELELK

SEQ ID NO: 91 - DNA, 345 bp, Artificial Sequence
  Mouse-human chimeric antibody H chain
caggtgcagctggtggagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgattaccgcggacgaatccacgagcacagcctacatggagctgagcagcctgagatctgaggacacggccgtgtattactgtgcgagattctactcctatacttactggggccagggaaccctggtcaccgtctcctca

SEQ ID NO: 92 - DNA, 345 bp, Artificial Sequence
  Mouse-human chimeric antibody H chain
caggtgcagctggtggagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacgaatccacgagcacagcctacatggagctgagcagcctgagatctgaggacacggccgtgtattactgtacaagattctactcctatacttactggggccagggaaccctggtcaccgtctcctca

SEQ ID NO: 93 - DNA, 345 bp, Artificial Sequence
  Mouse-human chimeric antibody H chain
caggtgcagctggtggagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacaaatccacgagcacagcctacatggagctgagcagcctgagatctgaggacacggccgtgtattactgtacaagattctactcctatacttactggggccagggaaccctggtcaccgtctcctca

SEQ ID NO: 94 - DNA, 345 bp, Artificial Sequence
  Mouse-human chimeric antibody H chain
caggtgcagctggtggagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacaaatccacgagcacagcctacatggagctgagcagcctgacatctgaggacacggccgtgtattactgtacaagattctactcctatacttactggggccagggaaccctggtcaccgtctcctca

SEQ ID NO: 95 - DNA, 345 bp, Artificial Sequence
  Mouse-human chimeric antibody H chain
caggtgcagctggtgcagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacgaatccacgagcacagcctacatggagctgagcagcctgagatctgaggacacggccgtgtattactgtacaagattctactcctatacttactggggccagggaaccctggtcaccgtctcctca

SEQ ID NO: 96 - DNA, 345 bp, Artificial Sequence
  Mouse-human chimeric antibody H chain
caggtgcagctggtgcagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacaaatccacgagcacagcctacatggagctgagcagcctgagatctgaggacacggccgtgtattactgtacaagattctactcctatacttactggggccagggaaccctggtcaccgtctcctca

SEQ ID NO: 97 - DNA, 345 bp, Artificial Sequence
  Mouse-human chimeric antibody H chain
caggtgcagctggtgcagtctggagctgaggtgaagaagcctggggcctcagtgaaggtctcctgcaaggcttctggatacaccttcaccgactatgaaatgcactgggtgcgacaggcccctggacaagggcttgagtggatgggagctcttgatcctaaaactggtgatactgcctacagtcagaagttcaagggcagagtcacgctgaccgcggacaaatccacgagcacagcctacatggagctgagcagcctgacatctgaggacacggccgtgtattactgtacaagattctactcctatacttactggggccagggaaccctggtcaccgtctcctca

SEQ ID NO: 98 - protein, 115 aa, Artificial Sequence
  Mouse-human chimeric antibody H chain
QVQLVESGAEVKKPGASVKVSCKASGYTFTDYEMHWVRQAPGQGLEWMGALDPKTGDTAYSQKFKGRVTITADESTSTAYMELSSLRSEDTAVYYCARFYSYTYWGQGTLVTVSS

SEQ ID NO: 99 - protein, 115 aa, Artificial Sequence
  Mouse-human chimeric antibody H chain
QVQLVESGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 100 - protein, 115 aa, Artificial Sequence
  Mouse-human chimeric antibody H chain
QVQLVESGAEVKKPGASVKVSCKASGYTFTDYEMHWVRQAPGQGLEWMGALDPKTGDTAYSQKFKGRVTLTADKSTSTAYMELSSLRSEDTAVYYCTRFYSYTYWGQGTLVTVSS

SEQ ID NO: 101 - protein, 115 aa, Artificial Sequence
  Mouse-human chimeric antibody H chain
QVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 102 - protein, 115 aa, Artificial Sequence
  Mouse-human chimeric antibody H chain
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 103 - protein, 115 aa, Artificial Sequence
  Mouse-human chimeric antibody H chain
QVQLVQSGAEVKKPGASVKVSCKASGYTFTDYEMHWVRQAPGQGLEWMGALDPKTGDTAYSQKFKGRVTLTADKSTSTAYMELSSLRSEDTAVYYCTRFYSYTYWGQGTLVTVSS

SEQ ID NO: 104 - protein, 115 aa, Artificial Sequence
  Mouse-human chimeric antibody H chain
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 105 - DNA, 336 bp, Artificial Sequence
  Mouse-human chimeric antibody L chain
gatgttgtgatgactcagtctccactctccctgcccgtcacccctggagagccggcctccatctcctgcagatctagtcagagccttgtacacagtaatggaaacacctatttacattggtacctgcagaagccagggcagtctccacagctcctgatctataaagtttccaaccgattttctggggtccctgacaggttcagtggcagtggatcaggcacagattttacactgaaaatcagcagagtggaggctgaggatgttggggtttattactgctctcaaaatacacatgttcctcctacgtttggccaggggaccaagctggagatcaaa

SEQ ID NO: 106 - protein, 112 aa, Artificial Sequence
  Mouse-human chimeric antibody L chain
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 107 - DNA, 27 bp, Artificial Sequence
  Synthetic Primer
cttgtacacagtgacggaaacacctat

SEQ ID NO: 108 - DNA, 27 bp, Artificial Sequence
  Synthetic Primer
ataggtgtttccgtcactgtgtacaag

SEQ ID NO: 109 - protein, 16 aa, Artificial Sequence
  Mutant antibody L chain
RSSQSLVHSNANTYLH

SEQ ID NO: 110 - protein, 16 aa, Artificial Sequence
  Mutant antibody L chain
RSSQSLVHSNDNTYLH

SEQ ID NO: 111 - protein, 16 aa, Artificial Sequence
  Mutant antibody L chain
RSSQSLVHSNENTYLH

SEQ ID NO: 112 - protein, 16 aa, Artificial Sequence
  Mutant antibody L chain
RSSQSLVHSNFNTYLH

SEQ ID NO: 113 - protein, 16 aa, Artificial Sequence
  Mutant antibody L chain
RSSQSLVHSNHNTYLH

SEQ ID NO: 114 - protein, 16 aa, Artificial Sequence
  Mutant antibody L chain
RSSQSLVHSNNNTYLH

SEQ ID NO: 115 - protein, 16 aa, Artificial Sequence
  Mutant antibody L chain
RSSQSLVHSNTNTYLH

SEQ ID NO: 116 - protein, 16 aa, Artificial Sequence
  Mutant antibody L chain
RSSQSLVHSNQNTYLH

SEQ ID NO: 117 - protein, 17 aa, Artificial Sequence
  Mutant antibody L chain
RSSQSLVHSNGINTYLH

SEQ ID NO: 118 - protein, 16 aa, Artificial Sequence
  Mutant antibody L chain
RSSQSLVHSNKNTYLH

SEQ ID NO: 119 - protein, 16 aa, Artificial Sequence
  Mutant antibody L chain
RSSQSLVHSNLNTYLH

SEQ ID NO: 120 - protein, 16 aa, Artificial Sequence
  Mutant antibody L chain
RSSQSLVHSNSNTYLH

SEQ ID NO: 121 - protein, 16 aa, Artificial Sequence
  Mutant antibody L chain
RSSQSLVHSNWNTYLH

SEQ ID NO: 122 - protein, 16 aa, Artificial Sequence
  Mutant antibody L chain
RSSQSLVHSNYNTYLH

SEQ ID NO: 123 - protein, 16 aa, Artificial Sequence
  Mutant antibody L chain
RSSQSLVHSNRNTYLH

SEQ ID NO: 124 - protein, 16 aa, Artificial Sequence
  Mutant antibody L chain
RSSQSLVHSNVNTYLH

SEQ ID NO: 125 - protein, 16 aa, Artificial Sequence
  Mutant antibody L chain
RSSQSLVHSNPNTYLH

SEQ ID NO: 126 - protein, 112 aa, Artificial Sequence
  Mutant antibody L chain
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 127 - protein, 112 aa, Artificial Sequence
  Mutant antibody L chain
DVVMTQSPLSLPVTPGEPASISCRSSQSLVHSNDNTYLHWYLQKPGQSPQLLIYKVSNRFSGVPDRFSGSGSGTDFTLKISRVEAEDVGVYYCSQNTHVPPTFGQGTKLEIK

SEQ ID NO: 128 - protein, 112 aa, Artificial Sequence
  Mutant antibody L chain
DVVMTQSPLSLPVTPGEPASISCRSSQSLVHSNENTYLHWYLQKPGQSPQLLIYKVSNRFSGVPDRFSGSGSGTDFTLKISRVEAEDVGVYYCSQNTHVPPTFGQGTKLEIK

SEQ ID NO: 129 - protein, 112 aa, Artificial Sequence
  Mutant antibody L chain
DVVMTQSPLSLPVTPGEPASISCRSSQSLVHSNFNTYLHWYLQKPGQSPQLLIYKVSNRFSGVPDRFSGSGSGTDFTLKISRVEAEDVGVYYCSQNTHVPPTFGQGTKLEIK

SEQ ID NO: 130 - protein, 112 aa, Artificial Sequence
  Mutant antibody L chain
DVVMTQSPLSLPVTPGEPASISCRSSQSLVHSNHNTYLHWYLQKPGQSPQLLIYKVSNRFSGVPDRFSGSGSGTDFTLKISRVEAEDVGVYYCSQNTHVPPTFGQGTKLEIK

SEQ ID NO: 131 - protein, 112 aa, Artificial Sequence
  Mutant antibody L chain
DVVMTQSPLSLPVTPGEPASISCRSSQSLVHSNNNTYLHWYLQKPGQSPQLLIYKVSNRFSGVPDRFSGSGSGTDFTLKISRVEAEDVGVYYCSQNTHVPPTFGQGTKLEIK

SEQ ID NO: 132 - protein, 112 aa, Artificial Sequence
  Mutant antibody L chain
DVVMTQSPLSLPVTPGEPASISCRSSQSLVHSNTNTYLHWYLQKPGQSPQLLIYKVSNRFSGVPDRFSGSGSGTDFTLKISRVEAEDVGVYYCSQNTHVPPTFGQGTKLEIK

SEQ ID NO: 133 - protein, 112 aa, Artificial Sequence
  Mutant antibody L chain
DVVMTQSPLSLPVTPGEPASISCRSSQSLVHSNQNTYLHWYLQKPGQSPQLLIYKVSNRFSGVPDRFSGSGSGTDFTLKISRVEAEDVGVYYCSQNTHVPPTFGQGTKLEIK

SEQ ID NO: 134 - protein, 112 aa, Artificial Sequence
  Mutant antibody L chain
DVVMTQSPLSLPVTPGEPASISCRSSQSLVHSNINTYLHWYLQKPGQSPQLLIYKVSNRFSGVPDRFSGSGSGTDFTLKISRVEAEDVGVYYCSQNTHVPPTFGQGTKLEIK

SEQ ID NO: 135 - protein, 112 aa, Artificial Sequence
  Mutant antibody L chain
DVVMTQSPLSLPVTPGEPASISCRSSQSLVHSNKNTYLHWYLQKPGQSPQLLIYKVSNRFSGVPDRFSGSGSGTDFTLKISRVEAEDVGVYYCSQNTHVPPTFGQGTKLEIK

SEQ ID NO: 136 - protein, 112 aa, Artificial Sequence
  Mutant antibody L chain
DVVMTQSPLSLPVTPGEPASISCRSSQSLVHSNLNTYLHWYLQKPGQSPQLLIYKVSNRFSGVPDRFSGSGSGTDFTLKISRVEAEDVGVYYCSQNTHVPPTFGQGTKLEIK

SEQ ID NO: 137 - protein, 112 aa, Artificial Sequence
  Mutant antibody L chain
DVVMTQSPLSLPVTPGEPASISCRSSQSLVHSNSNTYLHWYLQKPGQSPQLLIYKVSNRFSGVPDRFSGSGSGTDFTLKISRVEAEDVGVYYCSQNTHVPPTFGQGTKLEIK

SEQ ID NO: 138 - protein, 112 aa, Artificial Sequence
  Mutant antibody L chain
DVVMTQSPLSLPVTPGEPASISCRSSQSLVHSNWNTYLHWYLQKPGQSPQLLIYKVSNRFSGVPDRFSGSGSGTDFTLKISRVEAEDVGVYYCSQNTHVPPTFGQGTKLEIK

SEQ ID NO: 139 - protein, 112 aa, Artificial Sequence
  Mutant antibody L chain
DVVMTQSPLSLPVTPGEPASISCRSSQSLVHSNYNTYLHWYLQKPGQSPQLLIYKVSNRFSGVPDRFSGSGSGTDFTLKISRVEAEDVGVYYCSQNTHVPPTFGQGTKLEIK

SEQ ID NO: 140 - protein, 112 aa, Artificial Sequence
  Mutant antibody L chain
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 141 - protein, 112 aa, Artificial Sequence
  Mutant antibody L chain
DVVMTQSPLSLPVTPGEPASISCRSSQSLVHSNVNTYLHWYLQKPGQSPQLLIYKVSNRFSGVPDRFSGSGSGTDFTLKISRVEAEDVGVYYCSQNTHVPPTFGQGTKLEIK

SEQ ID NO: 142 - protein, 112 aa, Artificial Sequence
  Mutant antibody L chain
DVVMTQSPLSLPVTPGEPASISCRSSQSLVHSNPNTYLHWYLQKPGQSPQLLIYKVSNRFSGVPDRFSGSGSGTDFTLKISRVEAEDVGVYYCSQNTHVPPTFGQGTKLEIK

(19) United States
(12) Patent Application Publication
(10) Pub. No.: US 2008/0166756 A1
(43) Pub. Date: Jul. 10, 2008

(54) Method of Producing an Antibody Using a Cell in Which the Function of Fucose Transporter Is Inhibited

(76) Inventors: Masayuki Tsuchiya, Shizuoka (JP); Shigeyuki IIjima, Shizuoka (JP); Izumi Sugo, Shizuoka (JP); Yasuo Sekimori, Shizuoka (JP); Kiyoshi Habu, Shizuoka (JP); Masamichi Sugimoto, Kanagawa (JP)

Correspondence Address: BIRCH STEWART KOLASCH & BIRCH, PO BOX 747, FALLS CHURCH, VA, 22040-0747, US

(21) Appl. No.: 11/793,649

(22) PCT Filed: Oct. 26, 2005
(86) PCT No.: PCT/JP2005/020060

§ 371 Date: Jun. 21, 2007

(30) Foreign Application Priority Data
Dec. 22, 2004 (JP) PCT JP2004 019261

(51) Int. Cl. C12P 21/04; C12N 5/06; C12N 15/87

(52) U.S. Cl. 435/69.1; 435/325; 435/358; 435/69.6; 435/440

(57) ABSTRACT

The present invention relates to a method of producing a recombinant protein particularly an antibody using a cell in which the function of a fucose transporter is inhibited. According to the present invention, a cell in which the expression of fucose transporter genes on both homologous chromosomes is artificially suppressed is provided.

### TECHNICAL FIELD

The present invention relates to a method of producing a recombinant protein, particularly an antibody, using a cell in which the function of a fucose transporter is inhibited.

### BACKGROUND ART

Antibodies can exert anti-tumor effects via their ADCC (antibody-dependent cell-mediated cytotoxicity) activity or CDC (complement dependent cytotoxicity) activity. Antibodies are sugar chain-bound glycoproteins. It is known that an antibody's cytotoxic activity level can vary depending on the types and amounts of sugar chains that bind to the antibody. In particular, it has been reported that the amount of fucose binding to an antibody is strongly involved in the cytotoxic activity level (Shields et al., J Biol Chem., 277(30), 26733-26740, 2002). Furthermore, a method of producing a recombinant antibody not having fucose has been reported. Such method involves preventing an enzyme that catalyzes the binding of fucose to a sugar chain from being expressed upon antibody production in order to obtain an antibody with enhanced cytotoxic activity (International Patent Publication No. WO00/61739).

### DISCLOSURE OF THE INVENTION

An object of the present invention is to provide a method for easily and reliably producing a recombinant protein wherein the binding of fucose is eliminated or decreases. In particular, an object of the present invention is to provide a method of producing an antibody wherein the binding of fucose is eliminated or decreases and whose cytotoxic activity is enhanced. Furthermore, another object of the present invention is to provide a host cell for producing such protein.

### MEANS TO SOLVE THE PROBLEMS

In a mechanism by which fucose binds to an antibody within an antibody-producing cell, it is known that GDP binds to fucose that has been incorporated into a cell. GDP-fucose is then incorporated into the Golgi apparatus and then the fucose of the GDP-fucose is transferred to N-acetylglucosamine that has been added as a sugar chain to protein within the Golgi apparatus. Specifically, the Fc region of an antibody molecule has two sites to which an N-glycoside-bound sugar chain binds. Fucose binds to the N-acetylglucosamine portion of an N-glycoside-bound sugar chain (Pate L. Smith et al., J. Cell Biol. 2002, 158, 801-815).

The present inventors have considered that disruption of fucose transporter genes on both chromosomes leads to inhibition of the incorporation of fucose into the Golgi apparatus, so that addition of fucose to an antibody can be inhibited. The present inventors have prepared a cell wherein fucose transporter genes on both chromosomes are disrupted and thus completed the present invention.

In the present invention, to satisfy conditions where the addition of fucose to an antibody is inhibited, it is not necessary that all the produced antibodies do not experience the addition of fucose thereto, but the proportion of protein to which fucose has been added should be decreased among antibody compositions.

The present invention will be described as follows.

[1] A cell, in which the expression of fucose transporter genes on both chromosomes is artificially suppressed.
[2] The cell according to [1], in which the fucose transporter genes are disrupted.
[3] The cell according to [1] or [2], which is an animal cell.
[4] The cell according to [3], in which the animal cell is a Chinese hamster cell.
[5] The cell according to [4], in which the animal cell is a CHO cell.
[6] The cell according to any one of [2] to [5], in which gene disruption is carried out by homologous recombination using a gene targeting vector.
[7] The cell according to any one of [1] to [6], in which a gene encoding a foreign protein is introduced.
[8] The cell according to [7], in which the gene encoding the foreign protein is a gene encoding an antibody.
[9] A method of producing a protein, comprising culturing the cell according to any one of [1] to [8].
[10] The production method according to [9], in which the protein is an antibody.
[11] The production method according to [10], comprising producing a protein to which fucose is not added.
[12] A method for inhibiting the addition of fucose to a protein, comprising artificially suppressing the expression of fucose transporter genes on both chromosomes upon production of a recombinant protein using a cell.
[13] The method for inhibiting the addition of fucose to a protein according to [12], comprising artificially suppressing gene expression through deletion of a gene.
[14] The method for inhibiting the addition of fucose to a protein according to [12] or [13], in which the protein is an antibody.
[15] The method for inhibiting the addition of fucose to a protein according to any one of [12] to [14], in which the cell is a CHO cell.

This description includes part or all of the contents as disclosed in the description and/or drawings of PCT International Patent Application No. PCT/JP2004/019261, which is a priority document of the present application.

### BRIEF DESCRIPTION OF THE DRAWINGS

FIG. 1 shows the structures of 3 types of targeting vector.

FIG. 2 shows the structures of bands that appeared after knockout of the first step and the following cleavage with Bgl II.

FIG. 3 shows the results of southern blot analysis in the case of the knockout of the first step.

FIG. 4 shows homologous recombination efficiencies in the case of the knockout of the second step.

FIG. 5 is a photograph showing the results of Southern blot analysis of fucose transporter gene-deficient cell lines.

FIG. 6 shows the results of fucose expression analysis of a fucose transporter gene-deficient cell line (wild/KO).

FIG. 7 shows the results of fucose expression analysis of a fucose transporter gene-deficient cell line (KO/KO).

FIG. 8 shows the ADCC activity of each anti-HM1.24 antibody as determined using human PBMC and ARH-77 as a target cell.

FIG. 9 shows the ADCC activity of each anti-GPC3 antibody as determined using human PBMC and HuH-7 as a target cell.

FIG. 10 shows the normal phase HPLC chromatograms of 2-AB-labeled sugar chains prepared from a humanized anti-HM1.24 antibody (a) produced by FT-KO cells and a humanized anti-HM1.24 antibody (b) produced by CHO cells.

FIG. 11 shows the normal phase HPLC chromatograms of agalactosyl 2-AB-labeled sugar chains prepared from humanized anti-GPC3 antibodies (a, b, and c) produced by FT-KO cells and a humanized anti-GPC3 antibody produced by CHO cells.

FIG. 12 shows inferred structures of peak G(0) and G(0)-Fuc shown in FIG. 11.

FIG. 13 shows a DSC (differential scanning calorimeter) measurement chart showing the results of measuring an antibody (a) produced by FT-KO cells and an antibody (b) produced by CHO cells.

### PREFERRED EMBODIMENTS OF THE INVENTION

“Fucose transporter” in the present invention means a polypeptide having fucose transport activity. For example, when a fucose transporter is expressed on the cell membrane, it generally incorporates fucose into the cells. When a fucose transporter is expressed on the Golgi membrane, it generally incorporates fucose into the Golgi apparatus. In the present invention, a preferable fucose transporter is a Chinese hamster fucose transporter, and a more preferable example is a fucose transporter having the amino acid sequence represented by SEQ ID NO: 2. SEQ ID NO: 1 shows the nucleotide sequence of the Chinese hamster fucose transporter gene.

The Golgi apparatus incorporates fucose into itself mainly via fucose transporters existing on the Golgi membrane. Through inhibition of the fucose transporter function, incorporation of fucose into the Golgi apparatus can be inhibited and the amount of fucose to be incorporated into the Golgi apparatus can be decreased.

To inhibit the fucose transporter function of cells means to cause a decrease or disappearance of the fucose transport activity of a fucose transporter.

The fucose transporter function of cells may be inhibited by any method. A method for inhibiting fucose transporter expression is preferable.

A method for inhibiting fucose transporter expression is not specifically limited, as long as the number of fucose transporters having normal transport ability decreases. A method that involves deleting (knockout) a gene (fucose transporter gene) encoding a fucose transporter through the use of a targeting vector targeting the fucose transporter or the like is preferable.

Generally a cell has fucose transporter genes on both chromosomes. The cell of the present invention, in which the function of a fucose transporter is inhibited, lacks fucose transporter genes on both chromosomes. Alternatively, the cell of the present invention, in which the function of a fucose transporter is inhibited, is not particularly limited, as long as it lacks two fucose transporter genes on both chromosomes. Preferably, the cell lacks whole fucose transporter genes existing on chromosomes. For example, when 3 fucose transporter genes in total exist on chromosomes, deletion of 3 fucose transporter genes is preferable. Whether or not all the fucose transporter genes on chromosomes have been deleted can be examined using Southern blot analysis as described in Examples below or the FISH method, for example.

Protein produced by the production method of the present invention may be any protein. In general, such protein is a glycoprotein and preferably an antibody.

Types of antibody that are produced by the method of the present invention are not specifically limited. For example, mouse antibodies, rat antibodies, rabbit antibodies, sheep antibodies, camel antibodies, human antibodies, and artificially altered (for the purpose of, for example, lowering heterologous antigenicity against humans) gene recombinant antibody such as a chimeric antibody or a humanized antibody can be appropriately used. Such gene recombinant antibodies can be produced using a known method. A chimeric antibody comprises the variable region of the heavy and light chains of an antibody of a non-human mammal such as a mouse and the constant region of the heavy and light chains of a human antibody. DNA encoding the variable region of a mouse antibody is ligated to DNA encoding the constant region of a human antibody. The resultant is incorporated into an expression vector, and then the vector is introduced into a host to cause the host to produce the gene product. Thus a gene recombinant antibody can be obtained. A humanized antibody is also referred to as a reshaped human antibody. A humanized antibody is obtained by transplanting the complementarity determining region (CDR) of an antibody of a non-human mammal such as a mouse into the complementarity determining region of a human antibody. General gene recombination techniques therefor are also known. Specifically, DNA sequences designed to have the CDR of a mouse antibody ligated to the framework region (FR) of a human antibody are synthesized by the PCR method from several oligonucleotides, adjacent oligonucleotides of which have an overlap region at their terminal portions. The thus obtained DNA is ligated to DNA encoding the constant region of a human antibody, the resultant is incorporated into an expression vector, and then the vector is introduced into a host to cause the host to produce the gene product, so that a gene recombinant antibody can be obtained (see European Patent Application Publication No. EP 239400 and International Patent Application Publication No. WO 96/02576). As the FR of a human antibody, which is ligated via CDR, FR that allows the formation of an antigen-binding site with a good complementarity determining region is selected. If necessary, for the formation of an antigen-binding site having the appropriate complementarity determining region of a reshaped human antibody, the amino acids of the framework region of an antibody variable region may be substituted (Sato, K. et al., Cancer Res, 1993, 53, 851-856.). Furthermore, methods for obtaining human antibodies are also known. For example, a desired human antibody having activity of binding to an antigen can also be obtained by sensitizing a human lymphocyte in vitro with a desired antigen or a cell that expresses a desired antigen and then fusing the sensitized lymphocyte to a human myeloma cell such as U266 (see JP Patent Publication (Kokoku) No. 1-59878 B (1989)). Moreover, a desired human antibody can be obtained by immunizing a transgenic animal that has all the repertories of a human antibody gene with a desired antigen (see International Patent Application Publication Nos. WO93/12227, WO92/03918, WO94/02602, WO94/25585, WO96/34096, and WO96/33735). Furthermore, a technique is also known by which a human antibody is obtained by panning using a human antibody library. For example, the variable region of a human antibody is expressed as a single chain antibody (scFv) on the surface of a phage by a phage display method. A phage that binds to an antigen can be selected. A DNA sequence encoding the variable region of a human antibody that binds to the antigen can be determined by analyzing the gene of the thus selected phage. When the DNA sequence of scFv that binds to an antigen is revealed, an appropriate expression vector is constructed based on the sequence, and then a human antibody can be obtained. These methods are already known. Concerning these methods, WO92/01047, WO92/20791, WO93/06213, WO93/11236, WO93/19172, WO95/01438, and WO95/15388 can be referred to.

Furthermore, the antibody of the present invention may be a lower molecular weight antibody such as an antibody fragment or a modified product of the antibody, as long as such antibody can bind to an antigen. Examples of such antibody fragment include Fab, F(ab′)2, Fv, or single chain Fv(scFv) wherein Fv of the H chain and Fv of the L chain are linked using an appropriate linker (Huston, J. S. et al., Proc. Natl. Acad. Sci. U.S.A. (1988) 85, 5879-5883), and a diabody. To obtain such antibody fragment, a gene encoding such antibody fragment is constructed, the gene is introduced into an expression vector, and then the gene is expressed in an appropriate host cell (e.g., see Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; and Bird, R. E. and Walker, B. W., Trends Biotechnol. (1991) 9, 132-137). A diabody is prepared by dimerization; specifically, by linking two fragments (e.g., scFv), each of which is prepared by linking two variable regions using a linker or the like (hereinafter, referred to as a fragment composing a diabody). Generally, a diabody contains two VLs and two VHs (P. Holliger et al., Proc. Natl. Acad. Sci. U.S.A. 90, 6444-6448 (1993); EP404097; WO93/11161; Johnson et al., Methods in Enzymology, 203, 88-98, (1991); Holliger et al., Protein Engineering, 9, 299-305, (1996); Perisic et al., Structure, 2, 1217-1226, (1994); John et al., Protein Engineering, 12(7), 597-604, (1999); Holliger et al., Proc. Natl. Acad. Sci. U.S.A. 90, 6444-6448, (1993); and Atwell et al., Mol. Immunol. 33, 1301-1312, (1996)).

As a modified product of an antibody, antibodies to which various molecules such as polyethylene glycol (PEG) have been bound can also be used. Furthermore, a radioactive isotope, a chemical therapeutic agent, a cytotoxic substance such as toxin derived from bacteria, or the like can be bound to an antibody. In particular, a radiolabeled antibody is useful. Such modified product of an antibody can be obtained by chemically modifying an obtained antibody. In addition, methods for modifying antibodies have already been established in the field.

### <Protein Production Method According to the Method of the Present Invention>

A recombinant polypeptide can be produced by a method known by persons skilled in the art. In general, such recombinant polypeptide can be purified and prepared as follows. DNA encoding a polypeptide is incorporated into an appropriate expression vector, a transformant that has been obtained by introducing the vector into an appropriate host cell is collected, and then an extract is obtained. Subsequently, the resultant is subjected to chromatography such as ion exchange chromatography, reverse phase chromatography, or gel filtration, affinity chromatography using a column (to which an antibody against the polypeptide of the present invention has been immobilized), or chromatography using combination of a plurality of such columns.

Furthermore, when protein is expressed as a fusion polypeptide with a glutathione-S-transferase protein or as a recombinant polypeptide to which a plurality of histidines have been added in host cells (e.g., animal cells or Escherichia coli), the expressed recombinant polypeptide can be purified using a glutathione column or a nickel column. After purification of the fusion polypeptide, if necessary, regions other than the target polypeptide can also be cleaved and removed from the fusion polypeptide using thrombin, factor Xa or the like.

Protein to be produced by the production method of the present invention is preferably an antibody with cytotoxic activity that is affected by fucose binding thereto.

A method of producing an antibody using genetic recombination techniques, which is well known by persons skilled in the art, involves incorporating an antibody gene into an appropriate vector, introducing the vector into a host, and thus causing the production of the antibody using genetic recombination techniques (e.g., see Carl, A. K. Borrebaeck, James, W. Larrick, THERAPEUTIC MONOCLONAL ANTIBODIES, Published in the United Kingdom by MACMILLAN PUBLISHERS LTD, 1990).

### <Cells to be Used in the Protein Production Method of the Present Invention and Protein Production Using the Cells>

Furthermore, the present invention encompasses a host cell that can produce a foreign protein, wherein the expression of fucose transporter genes existing on both chromosomes is artificially suppressed.

A protein having no fucose binding thereto can be obtained by expressing a foreign protein using the aforementioned cells wherein the expression of fucose transporter genes on both chromosomes is artificially suppressed as host cells. Here, a foreign protein means a protein not derived from the cell itself. Host cells are not specifically limited. For example, cells wherein sugar is added to a recombinant protein when the protein is expressed can be used. More specifically, various animal cells or the like can be used. Preferably CHO cells can be used. In the present invention, in particular, CHO cells wherein a fucose transporter gene has been knocked out can be appropriately used. As animal cells, mammalian cells such as CHO (J. Exp. Med. (1995) 108, 945), COS, 3T3, myeloma, BHK (baby hamster kidney), HeLa, and Vero are known. As amphibian cells, Xenopus oocytes (Valle, et al., Nature (1981) 291, 358-340), for example, are known. As insect cells Sf9, Sf21, and Tn5, for example, are known. Examples of CHO cells include dhfr-CHO cells (Proc. Natl. Acad. Sci. U.S.A. (1980) 77, 4216-4220) and CHO K-1 cells (Proc. Natl. Acad. Sci. U.S.A. (1968) 60, 1275), which are deficient in a DHFR gene. For the purpose of mass-expression in animal cells, CHO cells are particularly preferable.

Protein having no fucose binding thereto can be obtained by incorporating a gene encoding a foreign protein such as an antibody to be produced into an expression vector and then incorporating the expression vector into host cells capable of producing such foreign protein; that is, cells having an inhibited fucose transporter function. Examples of vectors include expression vectors derived from mammals (e.g., pcDNA3 (produced by Invitrogen Corporation) and pEGF-BOS (Nucleic Acids. Res. 1990, 18(17), p. 5322), pEF, and pCDM8), expression vectors derived from insect cells (e.g., the “Bac-to-BAC baculovirus expression system” (produced by GIBCO-BRL Life Technologies Inc.) and pBacPAK8), expression vectors derived from plants (e.g., pMH1 and pMH2), expression vectors derived from animal viruses (e.g., pHSV, pMV, and pAdexLcw), expression vectors derived from retroviruses (e.g., pZIPneo), expression vectors derived from yeast (e.g., the “Pichia Expression Kit” (produced by Invitrogen Corporation), pNV11, and SP-Q01), and expression vectors derived from Bacillus subtilis (e.g., pPL608 and pKTH50). When CHO cells are used as host cells, it is preferable to use a vector derived from a mammal.

For the purpose of expression in animal cells such as CHO cells, COS cells, or NIH3T3 cells, generally a vector used herein has a promoter required for expression within cells, such as an SV40 promoter (Mulligan et al., Nature (1979) 277, 108), an MMLV-LTR promoter, an EF1α promoter (Mizushima et al., Nucleic Acids Res. (1990) 18, 5322), or a CMV promoter. It is further preferable that such vector has a gene for selection of transformed cells (e.g., a drug resistance gene that enables distinguishment by the use of a drug (e.g., neomycin and G418)). Examples of a vector having such properties include pMAM, pDR2, pBK-RSV, pBK-CMV, pOPRSV, and pOP13.

Furthermore, an example of a method for the purpose of stable expression of a gene and amplification of the number of copies of a gene within cells involves introducing a vector (e.g., pCHOI) having a complementary DHFR gene into CHO cells that are deficient in the nucleic acid synthesis pathway, followed by amplification using methotrexate (MTX). Furthermore, an example of a method for the purpose of transient expression of a gene involves transforming COS cells having a gene that expresses SV40 T antigen on a chromosome with a vector (e.g., pcD) having an SV40 replication origin. As a replication initiation site, a site derived from polyoma virus, adenovirus, bovine papilloma virus (BPV), or the like can also be used. Furthermore, to amplify the number of copies of a gene in a host cell system, an expression vector may contain as a selection marker an aminoglycoside transferase (APH) gene, thymidine kinase (TK) gene, Escherichia coli xanthine-guanine phosphoribosyltransferase (Ecogpt) gene, dihydrofolate reductase (dhfr) gene, or the like.

A vector can be introduced into a host cell by, for example, a calcium phosphate method, a DEAE dextran method, a method using cationic ribosome DOTAP (produced by Boehringer Mannheim), an electroporation method, or lipofection.

Cell culture can be carried out according to a known method. As a culture solution for animal cells, DMEM, MEM, RPMI1640, or IMDM, for example, can be used. At this time, a serum fluid such as fetal calf serum (FCS) can be used together therewith. Alternatively serum-free culture may also be carried out. pH during culture is preferably between approximately 6 and 8. Culture is generally carried out at approximately 30° C. to 40° C. for approximately 15 to 200 hours. If necessary, exchange of media, aeration, and agitation are carried out.

An example of such a cell wherein the expression of fucose transporter genes on both chromosomes is artificially suppressed is a cell wherein fucose transporter genes existing on both chromosomes have been disrupted.

“Disruption of a gene” means the suppression of the expression of the gene by partial deletion, substitution, insertion, addition, or the like conducted for the nucleotide sequence of the gene. Here, “suppression of gene expression” may also include a case where the gene itself is expressed, but a gene encoding a protein having normal functions is not expressed.

“Disruption of a gene” of the present invention includes not only a case where gene expression is completely suppressed, but also a case wherein gene expression is partially suppressed. “Deletion (knockout) of a gene” and “inactivation of a gene” are also used so as to have a meaning equivalent to that of “disruption of a gene.” Furthermore, cells having a gene disrupted by homologous recombination using gene targeting are referred to as gene-knock out cells. A cell wherein a gene encoding a fucose transporter is disrupted is an example of a cell wherein fucose transporter expression is artificially suppressed.

A cell wherein a gene encoding a fucose transporter is disrupted is a cell wherein the amount of fucose existing in the Golgi apparatus is significantly decreased compared with that in a cell wherein a fucose transporter gene is not disrupted, a cell wherein intracellular fucose transport ability is lowered or deleted, or a cell wherein intracellular activity to incorporate fucose into the Golgi apparatus is lowered or eliminated.

In particular, cells wherein both fucose transporter genes homologous chromosome pair have been deleted exert the above properties more significantly than cells lacking single fucose transporter gene.

The amount of fucose in the Golgi apparatus can be measured by isolating the Golgi apparatus from a cell, extracting sugar, and then carrying out an antigen antibody reaction, a binding reaction between sugar and lectin, liquid chromatography, electrophoresis, or the like. Moreover, intracellular fucose transport ability and intracellular activity to incorporate fucose into the Golgi apparatus can be determined by, for example, using fucose labeled with a fluorescent substance, a radioisotope, or the like.

A gene can be disrupted by, for example, a homologous recombination method.

Such homologous recombination method means a method by which only the target gene is arbitrarily altered by homologous gene recombination between a gene on a chromosome and a foreign DNA. Another DNA sequence is inserted into an exon of a gene for the purpose of dividing a sequence encoding a protein. To facilitate identification of a cell having a gene targeting vector, a selection marker such as a neomycin resistance gene derived from a bacterium is generally used as a sequence to divide the gene. A targeting vector is designed and produced based on the sequence information of the fucose transporter gene described in this specification and the fucose transporter gene to be disrupted is then subjected to homologous recombination using the targeting vector. For example, a substitution vector can contain a homologous region that has been ligated to the 5′ and the 3′ side of mutation to be introduced, a positive selection marker, a restriction enzyme site for linearizing the vector outside the homologous region, a negative selection marker arranged outside the homologous region, a restriction enzyme cleavage site for detecting mutation, and the like. Targeting vectors can be produced according to methods described in, for example, edited by Kenichi Yamamura et al., Transgenic Animal, KYORITSU SHUPPAN CO., LTD., Mar. 1, 1997; Shinichi Aizawa, Gene Targeting, Production of Mutant Mice using ES cells, Bio Manual Series 8, YODOSHA CO., LTD., 1995; Hogan et al., Manipulating the Mouse Embryo, Cold Spring Harbor Laboratory Press (1944); Joyner, A. L., Gene Targeting, A Practical Approach Series, IRL Press (1993); and edited by Masami Matsumura et al., Experimental Medicine, Separate Volume, New Genetic Engineering Handbook (3rd revised version), YODOSHA CO., LTD., 1999. Both insertion and substitution targeting vectors may be used. Furthermore, recombination can also be caused by targeting using a Cre-lox system. Targeting using the Cre-lox system can be carried out according to a method described in, for example, JP Patent Publication (Kohyo) NO. 11-503015 A (1999). As a method for selecting homologous recombinants that have experienced homologous recombination, a known selection method such as positive selection, promoter selection, negative selection, or polyA selection may be used. For identification of a homologous recombinant, both the PCR method and the Southern blotting method can be used.

Furthermore, a cell wherein the fucose transporter gene of the present invention is disrupted can also be obtained by randomly introducing a mutation into a cell, as long as both fructose transporters on chromosome pair are deleted. Examples of a method for randomly introducing mutation into a cell include a method that involves randomly introducing a gene disruption vector containing a marker into the genome of a cell and then screening for a cell having a disrupted fucose transporter gene, and a method that involves randomly introducing mutation using an chemical mutagen such as ENU (N-ethyl-N-nitrosourea) and then screening for such cell having a disrupted fucose transporter gene. Screening for cells that have become unable to produce any fucose transporter may be carried out using fucose transporter activity as an index. Alternatively, such screening can also be carried out by Western blotting or Northern blotting using fucose transporter gene transcription or expression as an index.

Furthermore, the cell of the present invention having a disrupted fucose transporter gene can also be obtained from an animal having a knocked-out fucose transporter gene. Such animal having a knocked-out fucose transporter gene can be produced by disrupting a fucose transporter of an ES cell by the above method and then producing from the ES cell according to, for example, a method disclosed in WO02/33054 Publication. Examples of animals that are used in this case include, but are not limited to, goats, pigs, sheep, cattle, mice, hamsters, and rats. Established cells having no fucose transporter genes can be obtained by producing such established cells from animals having a knocked-out fucose transporter gene.

When a foreign recombinant protein is produced according to the present invention in host cells wherein two fucose transporter genes on both chromosomes have been disrupted, addition of fucose to a protein is inhibited. This is because fucose within each of the cells is not transported into the Golgi apparatus. In the case of such recombinant protein produced in host cells having disrupted fucose transporter genes, the amount of bound fucose is significantly lower or preferably unable to be detected, compared with the case of recombinant protein produced in host cells having an undisrupted fucose transporter gene. When a foreign protein is an antibody, a product can be obtained wherein no fucose is binding to an N-glycoside-bound sugar chain binding to 2 sugar-chain-binding sites existing in 1 molecule of an antibody; that is, existing in the Fc region composed of 2H chains. Such antibody having no fucose binding thereto has enhanced cytotoxic activity. In addition, when an antibody for which the addition of fucose thereto is inhibited is produced using the cell of the present invention, it is not necessary that all the produced antibodies have not experienced the addition of fucose thereto. The proportion of protein to which fucose has been added in antibody compositions should be reduced.

For example, in the case of an antibody that is produced using the animal cell of the present invention having disrupted fucose transporter genes, 50% or more, preferably 70% or more, further preferably 85% or more, and particularly preferably 90% or more of all the sugar chains has not experienced the addition of fucose thereto.

Furthermore, the present invention also encompasses animals (excluding humans) wherein fucose transporter genes on both chromosomes are deleted. A recombinant polypeptide can be produced in vivo using such animals.

DNA encoding target protein is introduced into these animals, polypeptides are produced in vivo within the animals, and then the polypeptides are collected. The term “host” in the present invention encompasses these animals and the like. When animals are used, there are production systems using mammals and production systems using insects. As mammals, goats, pigs, sheep, mice, or cattle can be used (Vicki Glaser, SPECTRUM Biotechnology Applications, 1993).

For example, a target DNA is prepared in the form of a fusion gene with a gene encoding a polypeptide such as goat β casein that is uniquely produced in milk. Subsequently, a DNA fragment comprising the fusion gene is injected into a goat embryo and then the embryo is transplanted into a female goat. A target polypeptide can be obtained from milk that is produced by transgenic goats born from goats that have accepted such embryos or from the progenies of such transgenic goats. To increase the amount of milk containing polypeptides, which is produced by transgenic goats, an appropriate hormone may also be used for such transgenic goats (Ebert, K. M. et al., Bio/Technology (1994) 12, 699-702).

The thus obtained polypeptide can be isolated from within host cells or outside the cells (e.g., media) and then purified as a substantially pure and uniform polypeptide. To separate and purify polypeptides, separation and purification methods that are generally used for polypeptide purification may be employed and are not specifically limited. For example, polypeptides can be separated and purified by the use of appropriate selection and combination of a chromatography column, a filter, ultrafiltration, salting-out, solvent precipitation, solvent extraction, distillation, immunoprecipitation, SDS-polyacrylamide gel electrophoresis, an isoelectric focusing method, dialysis, recrystallization, and the like.

Examples of chromatography include affinity chromatography, ion exchange chromatography, hydrophobic chromatography, gel filtration, reverse phase chromatography, and adsorption chromatography (Strategies for Protein Purification and Characterization: A Laboratory Course Manual, Ed Daniel R. Marshak et al., Cold Spring Harbor Laboratory Press, 1996). These types of chromatography can be carried out using liquid-phase chromatography such as HPLC or FPLC.

In addition, when a proper protein modification enzyme is caused to act on polypeptides before or after purification, modification can be arbitrarily carried out or peptides can be partially removed. As protein modification enzymes, trypsin, chymotrypsin, lysylendopeptidase, protein kinase, and glucosidase, for example, are used.

A known sequence can also be used for a gene encoding the H chain or the L chain of an antibody that is produced by the production method of the present invention. Furthermore, such gene can also be obtained by a method known by persons skilled in the art. For example, a gene encoding an antibody can be cloned and obtained from a hybridoma producing a monoclonal antibody or can also be obtained from an antibody library. Hybridomas can be produced basically using a known technique as described below. Specifically, a hybridoma can be produced by using as a sensitizing antigen a desired antigen or a cell that expresses a desired antigen, carrying out immunization according to a general immunization method using such antigen, fusing the thus obtained immunocyte with a known parent cell by a general cell fusion method, and then screening for a monoclonal antibody-producing cell (hybridoma) by a general screening method. The cDNA of an antibody variable region (V region) is synthesized from the mRNA of the thus obtained hybridoma using reverse transcriptase. The cDNA is ligated to DNA encoding a desired antibody constant region (C region), so that a gene encoding the H chain or the L chain can be obtained. A sensitizing antigen upon immunization is not specifically limited. For example, a target full-length protein or partial peptide can be used. Antigens can be prepared by a method known by persons skilled in the art. For example, antigens can be prepared according to a method using baculovirus (e.g., WO98/46777). Hybridomas can be produced according to, for example, Milstein et al's method (Kohler, G., and Milstein, C., Methods Enzymol. 1981, 73, 3-46) or the like. When the immunogenicity of an antigen is low, such antigen is bound to a macromolecule having immunogenicity, such as albumin, and then immunization is carried out.

Regarding an antibody library, many antibody libraries are already known. In addition, a production method for an antibody library is known. Hence, persons skilled in the art can appropriately obtain an antibody library.

Antibodies that are expressed and produced as described above can be purified by a known method that is used for general protein purification. Antibodies can be separated and purified by appropriate selection or combination of, for example, an affinity column (e.g., protein A column), a chromatography column, a filter, ultrafiltration, salting-out, and dialysis (Antibodies: A Laboratory Manual, Ed Harlow and David Lane, Cold Spring Harbor Laboratory, 1988).

A known means can be used for measuring the antigen-binding activity of an antibody (Antibodies A Laboratory Manual, Ed Harlow, David Lane, Cold Spring Harbor Laboratory, 1988). For example, ELISA (enzyme-linked immunosorbent assay), EIA (enzyme-linked immunoassay), RIA (radioimmunoassay), or a fluorescent immunoassay can be used.

The sugar chain structure of protein produced using the cell of the present invention can be analyzed by a method described in a 2-dimensional sugar chain mapping method (Anal. Biochem, 171, 73 (1988); Biochemical Experimental Methods 23-Methods for Studying Glycoprotein Sugar Chains, edited by Reiko Takahashi, Center for Academic Publications Japan (1989)). Moreover, sugar chains can also be analyzed by mass spectrometry such as MALDI-TOF-MS.

Furthermore, the stability, such as the thermostability, of a protein having no fucose added thereto that is produced using a cell of the present invention is equivalent to that of a protein to which fucose has been added.

Cytotoxic Activity of Antibody

Antibodies produced by the method of the present invention have enhanced cytotoxic activity.

Examples of cytotoxic activity in the present invention include antibody-dependent cell-mediated cytotoxicity (ADCC) activity and complement-dependent cytotoxicity (CDC) activity. In the present invention, CDC activity means cytotoxic activity of a complement system. ADCC activity means activity to damage a target cell. Specifically, when a specific antibody attaches to a cell surface antigen of a target cell, an Fcγ receptor-retaining cell (e.g., an immunocyte) binds to the Fc portion of the antibody via an Fcγ receptor, damaging the target cell.

Whether or not an antibody has ADCC activity or has CDC activity can be determined by a known method (e.g., Current protocols in Immunology, Chapter 7, Immunologic studies in humans, Editor John E. Coligan et al., John Wiley & Sons, Inc., (1993)).

Specifically, effector cells, a complement solution, and target cells are prepared.

### (1) Preparation of Effector Cells

A spleen is extracted from a CBA/N mouse or the like, and then spleen cells are separated in an RPMI1640 medium (produced by GIBCO). After washing with the same medium containing 10% fetal bovine serum (FBS, produced by HyClone), the cells are prepared to a concentration of 5×10⁶/ml, thereby preparing effector cells.

### (2) Preparation of a Complement Solution

Baby Rabbit Complement (produced by CEDARLANE LABORATORIES LIMITED) is diluted 10-fold in a 10% FBS-containing medium (produced by GIBCO), thereby preparing a complement solution.

### (3) Preparation of Target Cells

Pancreatic cancer cell lines (e.g., AsPC-1 and Capan-2) are radiolabeled by culturing the cell lines with 0.2 mCi ⁵¹Cr-sodium chromate (produced by Amersham Pharmacia Biotech) in a 10% FBS-containing DMEM medium at 37° C. for 1 hour. After radiolabeling, the cells are washed three times in a 10% FBS-containing RPMI1640 medium and then prepared to a cell concentration of 2×10⁵/ml, thereby preparing target cells.

Subsequently, ADCC activity or CDC activity are determined. To determine ADCC activity, target cells and antibodies are added in amounts of 50 ml each to a 96-well U-bottomed plate (produced by Becton, Dickinson and Company) and are then allowed to react on ice for 15 minutes. Next, 100 μl of effector cells is added, followed by 4 hours of culture in a carbon dioxide gas incubator. The final antibody concentration is 0 or 10 μg/ml. After culture, 100 μl of the supernatant is collected, and then radioactivity is measured using a gamma counter (COBRAIIAUTO-GMMA, MODEL D5005, produced by Packard Instrument Company). Cytotoxic activity (%) can be calculated by (A−C)/(B−C)×100. “A” denotes radioactivity (cpm) in each sample, “B” denotes radioactivity (cpm) in a sample supplemented with 1% NP-40 (produced by NACALAI TESQUE, INC.), and “C” denotes radioactivity (cpm) in a sample containing only target cells.

Furthermore, to determine CDC activity, target cells and anti-PepT antibodies are added in amounts of 50 μl each to a 96-well flat-bottomed plate (produced by Becton, Dickinson and Company) and are then allowed to react on ice for 15 minutes. Subsequently, 100 μl of a complement solution is added, followed by 4 hours of culture in a carbon dioxide gas incubator. The final antibody concentration is 0 or 3 μg/ml. After culture, 100 μl of the supernatant is collected, and then radioactivity is measured using a gamma counter. Cytotoxic activity can be calculated in a manner similar to that used for determination of ADCC activity.

For example, the ADCC activity of an antibody produced using an animal cell of the present invention having a disrupted fucose transporter gene is compared with that of an antibody produced using an animal cell of the same species having an undisrupted fucose transporter gene. Comparison is made by finding antibody concentrations that lead to 50% cytotoxic activity from an antibody concentration-cytotoxic activity (%) curve. An antibody concentration that leads to 50% cytotoxic activity of an antibody that is produced using an animal cell having a disrupted fucose transporter gene is ½, preferably ⅕, and further preferably 1/10 of the antibody concentration that leads to 50% cytotoxicity of an antibody that is produced using an animal cell having an undisrupted fucose transporter gene. That is, the ADCC activity of an antibody produced using an animal cell of the present invention having a disrupted fucose transporter gene is enhanced to a level 2 or more, preferably 5 or more, and further preferably 10 or more times greater than that of an antibody produced using an animal cell having an undisrupted fucose transporter gene.

The present invention will be described in more detail below with reference to examples. However, the present invention is not limited to these examples.

### Example 1 Disruption of a Fucose Transporter Gene in CHO Cells

### 1. Construction of Targeting Vectors

### (1) Construction of a KO1 Vector

A hygromycin resistance gene (Hyg^{r}) was prepared by PCR using pcDNA3.1/Hygro (Invitrogen Corporation) as a template and Hyg5-BH and Hyg3-NT as primers. A BamH I and a TGCGC sequence were added to the 5′ side of the initiation codon of Hyg^{r}, thereby preparing a sequence that was the same as that on the 5′ region of the initiation codon of a fucose transporter gene. A Not I sequence was added to the 3′ region comprising a SV40 polyA addition signal, thereby extracting Hyg^{r}.

Forward primer
Hyg5-BH

(SEQ ID NO: 3)

5′-GGA TCC TGC GCA TGA AAA AGC CTG AAC TCA CC-3′
Reverse primer
Hyg3-NT

(SEQ ID NO: 4)

5′-GCG GCC GCC TAT TCC TTT GCC CTC GGA CG-3′

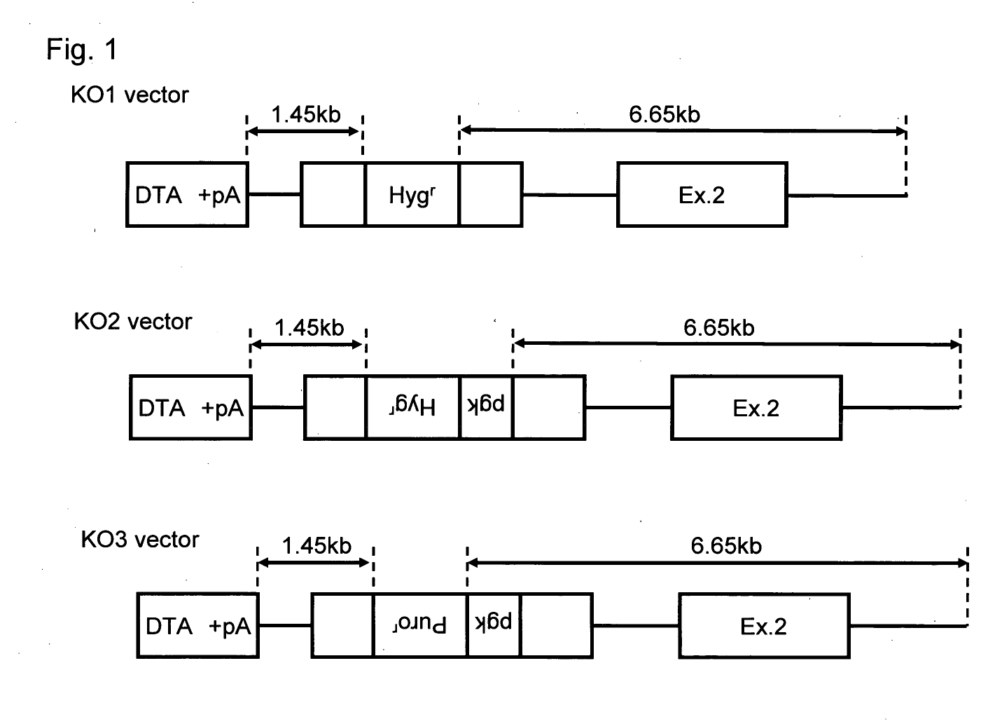

A targeting vector ver. 1 (hereinafter referred to as KO1 vector) for a fucose transporter knockout was constructed by inserting separately a 5′ fragment (ranging from No. 2,780 Sma I to No. 4,232 BamH I of the nucleotide sequence shown in SEQ ID NO: 1) of a fucose transporter, a 3′ fragment (ranging from No. 4,284 to No. 10,934 Sac I) of the fucose transporter, and a Hyg^{r} fragment into a pMC1DT-A vector (Yagi T, Proc. Natl. Acad. Sci. U.S.A. vol. 87, pp. 9918-9922, 1990) (FIG. 1). The vector is characterized by carrying promoter-less Hyg^{r} unit. Thus, Hyg^{r} is expressed by a promoter of the fucose transporter following homologous recombination. However, introduction of only one copy of a vector into cells by homologous recombination does not always result in the enough expression of Hyg^{r} that provide resistance against hygromycin B in the cells. In addition, the KO1 vector was linealized by Not I and transfected into cells. With the use of the KO1 vector, the fucose transporter gene will lack 41 base pairs of exon 1 comprising the initiation codon and lose the relevant function.

(2) Preparation of pBSK-pgk-1-Hyg^{r}

A mouse pgk-1 gene promoter was prepated from pKJ2 vector (Popo H, Biochemical Genetics vol. 28, pp. 299-308, 1990) by EcoR I-Pst I digestion and then cloned into the EcoR I-Pst I site of pBluescript (Stratagene Corporation), thereby preparing pBSK-pgk-1. A hygromycin resistance gene (Hyg^{r}) was prepared by PCR using pcDNA3.1/Hygro (Invitrogen Corporation) as a template and Hyg5-AV and Hyg3-BH as primers. Thus, an Eco T221 and a Kozak sequence were added to the 5′ region of Hyg^{r}. Furthermore, a BamH I sequence was added to the 3′ region comprising a SV40 polyA addition signal, thereby extracting Hyg^{r}.

Forward primer
Hyg5-AV

(SEQ ID NO: 5)

5′-ATG CAT GCC ACC ATG AAA AAG CCT GAA CTC ACC-3′
Reverse primer
Hyg3-BH

(SEQ ID NO: 6)

5′-GGA TCC CAG GCT TTA CAC TTT ATG CTT C-3′

The Hyg^{r} (EcoT22 I-BamH I) fragment was inserted into the Pst I-BamH I site of pBSK-pgk-1, thereby preparing pBSK-pgk-1-Hyg^{r}.

(3) Preparation of KO2 vector

A targeting vector ver.2 (hereinafter referred to as the KO2 vector) for a fucose transporter knockout was constructed by inserting separately a 5′region (ranging from No. 2,780 Sma I to No. 4,232 BamH I in the nucleotide sequence shown in SEQ ID NO: 1), a 3′region (ranging from No. 4,284 to No. 10,934 Sac I) of the fucose transporter, and pgk-1-Hyg^{r} fragments into a pMC1DT-A vector (FIG. 1). Unlike the KO1 vector, a pgk-1 gene promoter was added to Hyg^{r} of the KO2 vector. Introduction of even only one copy of a vector into cells via homologous recombination can cause the cells to acquire resistance to hygromycin B. In addition, the KO2 vector was linealized by Not I and then transfected into cells. With the use of the KO2 vector, the fucose transporter may lack 46 base pairs of exon 1 comprising the initiation codon and lose the relevant function.

(4) Preparation of pBSK-pgk-1-Puro^{r}

A pPUR vector (BD Biosciences) was digested with Pst I and BamH I. The Puro^{r} fragment was inserted into the Pst I-BamH I site of pBSK-pgk-1, thereby preparing pBSK-pgk-1-Puro^{r}.

### (5) Preparation of KO3 Vector

A targeting vector ver.3 (hereinafter referred to as the KO3 vector) for a fucose transporter knockout was constructed by separately inserting a 5′ region (ranging from No. 2,780 Sma I to No. 4,232 BamH I of the nucleotide sequence shown in SEQ ID NO: 1), a 3′ region (ranging from No. 4,284 to No. 10,934 Sac I) of the fucose transporter, and pgk-1-Puro^{r} fragments into a pMC1DT-A vector (FIG. 1). In addition, a sequence, to which the following primer for screening binds had been previously added to the 3′ end of pgk-1-Puro^{r}. In addition, the KO3 vector was linealized with Not I and then transfected into cells. With the use of the KO3 vector, the fucose transporter may lack 46 base pairs of exon 1 comprising the initiation codon and lose the relevant function.

Reverse primer
RSGR-A

(SEQ ID NO: 7)

5′-GCT GTC TGG AGT ACT GTG CAT CTG C-3′

Knockout of the fucose transporter gene was attempted using the above 3 types of targeting vector.

### 2. Vector Transfection into CHO Cells

HT Supplement (100×) (Invitrogen Corporation cat. 11067-030) and penicillin streptomycin (Invitrogen Corporation cat. 15140-122) were each added to CHO—S—SFMII HT-(Invitrogen Corporation cat 12052-098) in a volume one-hundredth of the volume of CHO—S—SFMII HT-. The solution was used as medium for culture (hereinafter referred to as SFMII (+)). The DXB11 cell line of CHO cells was maintained and cells after gene transfer were also cultured in SFMII (+). 8×10⁶ CHO cells were suspended in 0.8 mL of Dulbecco's phosphate buffer (hereinafter abbreviated as “PBS”; Invitrogen Corporation cat. 14190-144). 30 μg of the targeting vector was added to the cell suspension. The cell suspension was transferred to a Gene Pulser Cuvette (4 mm) (Bio-Rad Laboratories Inc. cat. 1652088). After the suspension had been allowed to stand on ice for 10 minutes, the cells were electroporated using GENE-PULSER II (Bio-Rad Laboratories Inc. code No. 340BR) at setting of 1.5 kV, 25 μFD. After vector transfection, the cells were suspended in 200 ml of SFMII(+) medium. The cell suspension was then dispensed into twenty 96-well flat-bottomed plates (IWAKI cat. 1860-096) at 100 μl/well. The cells in the plates were cultured in a CO₂ incubator for 24 hours at 37° C., followed by addition of a drug.

### 3. First Step of Knockout

The KO1 vector or the KO2 vector was transfected into CHO cells. At 24 hours after vector transfection, selection was performed using hygromycin B (Invitrogen Corporation cat. 10687-010). Hygromycin B was added to SFMII(+) to a concentration of 0.3 mg/ml and then added to transfected cells at 100 μl/well.

### 4. Screening of Homologous Recombinants by PCR

### (1) Preparation of Samples for PCR

Homologous recombinants were screened by the PCR method. CHO cells to be used in screening were cultured in a 96-well flat-bottomed plate. After removal of culture supernatants, a buffer for cell lysis was added at 50 μl/well. After incubation at 55° C. for 2 hours, proteinase K was inactivated by subsequent heating at 95° C. for 15 minutes, so as to prepare a template for PCR. The buffer for cell lysis was composed of 5 μl of 10×LA buffer II (attached to TaKaRa LA Taq), 2.5 μl of 10% NP-40 (Roche cat. 1 332 473), 4 μl of proteinase K (20 mg/ml and TaKaRa cat. 9033), and 38.5 μl of distilled water (Nacalai Tesque Inc. cat. 36421-35) per well.

### (2) PCR Conditions

A PCR reaction mixture was determined to contain 1 μl of each of the above PCR samples, 5 μl of 10×LA buffer II, 5 μl of MgCl₂ (25 mM), 5 μl of dNTP (2.5 mM), 2 μl of each primer (10 μM each), 0.5 μl of LA Taq (5 IU/μl and cat. RR002B), and 29.5 μl of distilled water (total 50 μl). For screening of the cells in which the KO1 vector had been transfected, TP-F4 and THygro-R1 were used as PCR primers. For screening of the cells in which the KO2 vector had been introduced, TP-F4 and THygro-F1 were used as PCR primers.

Moreover, PCR conditions employed for the cells into which the KO1 vector had been transfected consisted of pre-heating at 95° C. for 1 minute, 40 amplification cycles (each cycle consisting of 95° C. for 30 seconds, 60° C. for 30 seconds, and 60° C. for 2 minutes), and additional heating at 72° C. for 7 minutes. PCR conditions employed for screening of the cells into which the KO2 vector had been transfected consisted of pre-heating at 95° C. for 1 minute, 40 amplification cycles (each cycle consisting of 95° C. for 30 seconds and 70° C. for 3 minutes), and additional heating at 70° C. for 7 minutes.

Primers are as shown below. In cell samples of homologous recombinants, an approximately 1.6 kb DNA in the case of the KO1 vector and an approximately 2.0-kb DNA in the case of the KO2 vector were amplified. Regarding the primers, TP-F4 was set in the 5′ region of fucose transporter genome outside the vector. THygro-F1 and THygro-R1 were arranged within Hyg^{r} in the vector.

Forward primer (KO1, K02)

(SEQ ID NO: 8)

| | |
|---|---|
| TP-F4 | 5′-GGA ATG CAG CTT CCT CAA GGG ACT CGC-3′ |

Reverse primer (KO1)

(SEQ ID NO: 9)

| | |
|---|---|
| THygro-R1 | 5′-TGC ATC AGG TCG GAG ACG CTG TCG AAC-3′ |

Reverse primer (KO2)

(SEQ ID NO: 10)

| | |
|---|---|
| THygro-F1 | 5′-GCA CTC GTC CGA GGG CAA AGG AAT AGC-3′ |

### 5. PCR Screening Results

918 cells into which the KO1 vector had been transfected were analyzed. Of these cells, 1 cell was considered to be a homologous recombinant (with homologous recombination efficiency of approximately 0.1%). Furthermore, 537 cells into which the KO2 vector had been transfected were analyzed. Of these cells, 17 cells were considered to be homologous recombinants (with homologous recombination efficiency of approximately 3.2%).

### 6. Southern Blot Analysis

Such confirmation was also performed by the Southern blot method. 10 μg of genomic DNA was prepared from the cultured cells according to a standard method, and then Southern blot was performed. A 387-bp probe was prepared from the region ranging from No. 2,113 to No. 2,500 of the nucleotide sequence shown in SEQ ID NO: 1 by the PCR method using the following 2 primers. The thus prepared probe was used for confirmation by the Southern blot method. The genomic DNA was digested with Bgl II.

Forward primer

(SEQ ID NO: 11)

| | |
|---|---|
| Bgl-F: | 5′-TGT GCT GGG AAT TGA ACC CAG GAC-3′ |

Reverse primer

(SEQ ID NO: 12)

| | |
|---|---|
| Bgl-R: | 5′-CTA CTT GTC TGT GCT TTC TTC C-3′ |

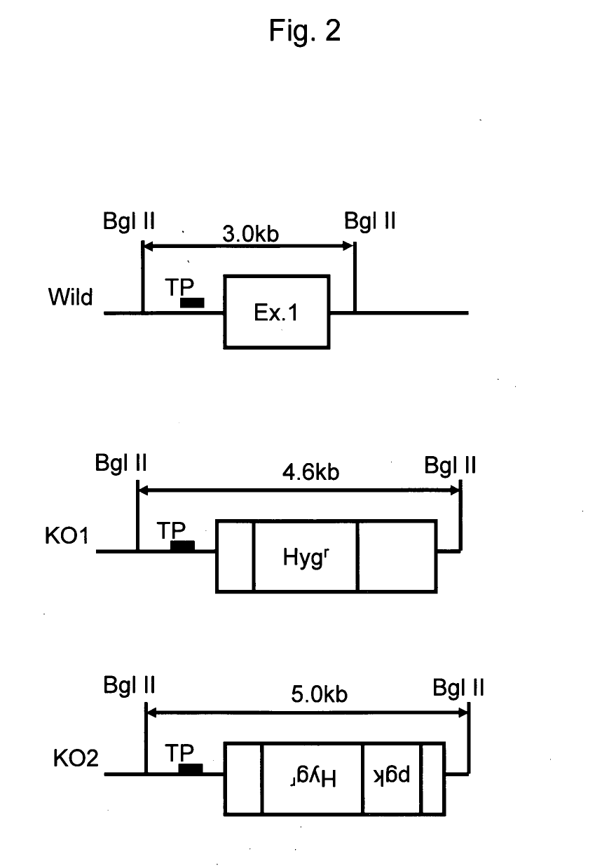

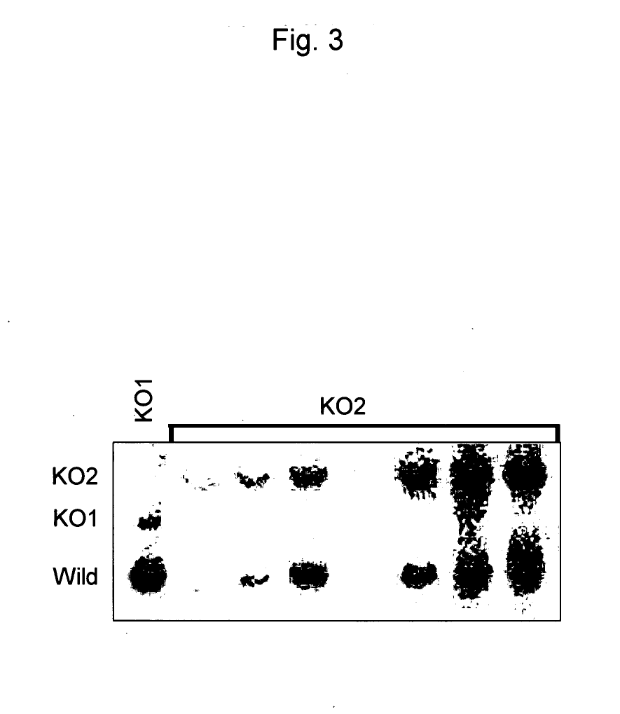

As a result of digestion with Bgl II, an approximately 3.0-kb band was detected from the chromosome of the fucose transporter, an approximately 4.6-kb band was detected from the chromosome into which the KO1 vector had been inserted after homologous recombination, and an approximately 5.0-kb band was detected from the chromosome into which the KO2 vector had been inserted after homologous recombination. FIG. 2 shows the band patterns after digestion with Bgl II following the knockout of the first step. FIG. 3 shows data of Southern blot. 1 cell line of homologous recombinants using the KO1 vector and 7 cell lines of homologous recombinants using the KO2 vector were used for this experiment. The only one cell line obtained from the KO1-vector transfection was named 5C1. Subsequent analysis revealed that this cell line is composed of multiple cell populations. Cloning was performed by the limiting dilution method for use in the subsequent experiments. Furthermore, the cell line obtained from the KO2-vector transfection was named 6E2.

### 7. Second Step of Knock Out

3 types of vector were used for homologous recombinants obtained from transfection of the KO1 vector or the KO2 vector, so as to attempt to establish cell lines completely lacking the fucose transporter gene. Combinations of such vectors with cells are as follows: method 1 (KO2 vector and 5C1 cells (KO1)); method 2 (KO2 vector and 6E2 cells (KO2)); and method 3 (KO3 vector and 6E2 cells (KO2)). The vectors were separately transfected into each cell type. At 24 hours after vector introduction, selection was initiated using hygromycin B or puromycin (Nacalai Tesque Inc., cat. 29455-12). Hygromycin B was used in the case of method 1 at a final concentration of 1 mg/ml and in the case of method 2 at a final concentration of 7 mg/ml. Further, in the case of method 3, hygromycin B was added to a final concentration of 0.15 mg/ml and puromycin was added to a final concentration of 8 μg/ml.

### 8. Screening of Homologous Recombinants by PCR

Samples were prepared as described above. Screening in the case of method 1 was performed by PCR to detect signals of homologous recombination by both KO1 vector and KO2 vector. For screening in the case of method 2, the following PCR primers were designed. TPS-F1 was set in the Nos. 3,924-to-3,950 region and SHygro-R1 was set in the Nos. 4,248-to-4,274 region of the nucleotide sequence shown in SEQ ID NO: 1. With the use of these PCR primers, a gene region of 350 bp of the fucose transporter, which is deficient due to the KO2 vector, is amplified. Therefore, for PCR screening in the case of method 2, cells in which such 350-bp region had not been amplified were determined to be cells completely lacking the fucose transporter gene. PCR conditions consisted of preheating at 95° C. for 1 minute, 35 amplification cycles (each cycle consisting of 95° C. for 30 seconds and 70° C. for 1 minute), and additional heating at 70° C. for 7 minutes.

Forward primer
TPS-F1:

(SEQ ID NO: 13)

5′-CTC GAC TCG TCC CTA TTA GGC AAC AGC-3′
Reverse primer
SHygro-R1:

(SEQ ID NO: 14)

5′-TCA GAG GCA GTG GAG CCT CCA GTC AGC-3′

In the case of method 3, TP-F4 was used as a forward primer and RSGR-A was used as a reverse primer. PCR conditions consisted of preheating at 95° C. for 1 minute, 35 amplification cycles (each cycle consisting of 95° C. for 30 seconds, 60° C. for 30 seconds, and 72° C. for 2 minutes), and additional heating at 72° C. for 7 minutes. In the cell samples of homologous recombinants in the case of the KO3 vector, an approximately 1.6-kb DNA was amplified. As a result of the PCR, homologous recombination by the KO3 vector was confirmed. It was also confirmed that the region of homologous recombination by the KO2 vector was remained.

### 9. Results of PCR Screening

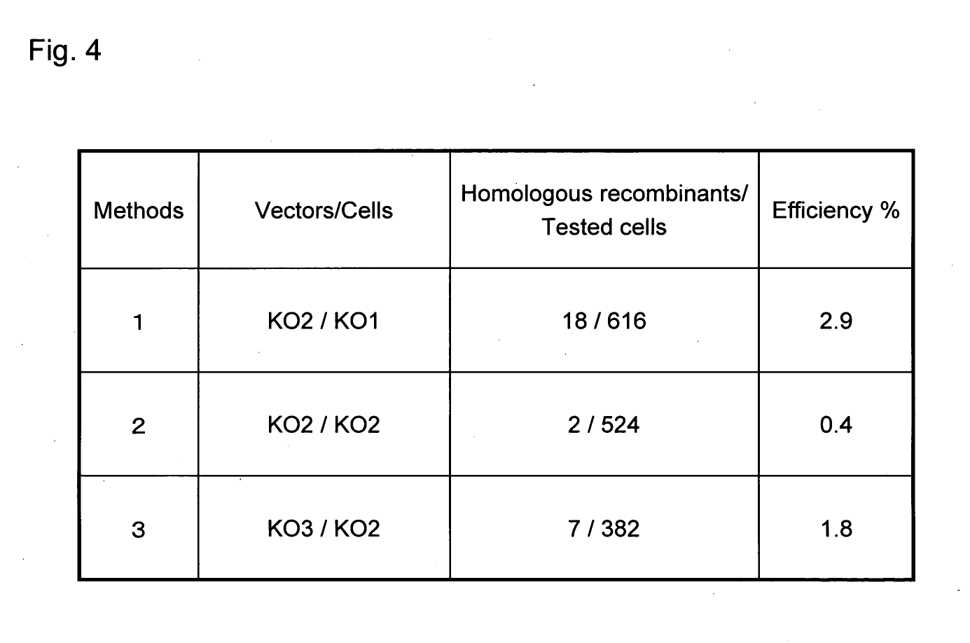

In the case of method 1, 616 cells were analyzed. Of these cells, 18 cells were thought to be homologous recombinants (homologous recombination efficiency: 2.9%) (FIG. 4). In the case of method 2, 524 cells were analyzed. Of these cells, 2 cells were thought to be homologous recombinants (homologous recombination efficiency: approximately 0.4%). Furthermore, in the case of method 3, 382 cells were analyzed. Of these cells, 7 cells were thought to be homologous recombinants (homologous recombination efficiency: approximately 1.8%).

### 10. Southern Blot Analysis

Southern blot analysis was performed according to the above methods. As a result, among the analyzed cells, one cell completely lacking the fucose transporter gene was discovered. In the case of knockout of the first step, PCR analysis results were consistent with Southern blot analysis results. Regarding knockout of the second step, the two were not consistent with each other. Possible causes of such results are: 1. a mixture of homologous recombinants of either KO1 alone or KO2 alone in the case of method 1; 2. presence of not a pair of fucose transporter genes (2 fucose transporter genes), but a plural number of pairs of fucose transporter genes (or 3 or more fucose transporter genes); and 3. increased copy number of the fucose transporter gene that had remained after subculture of cell lines in which the knockout of the first step had been successfully performed.

### 11. Fucose Expression Analysis

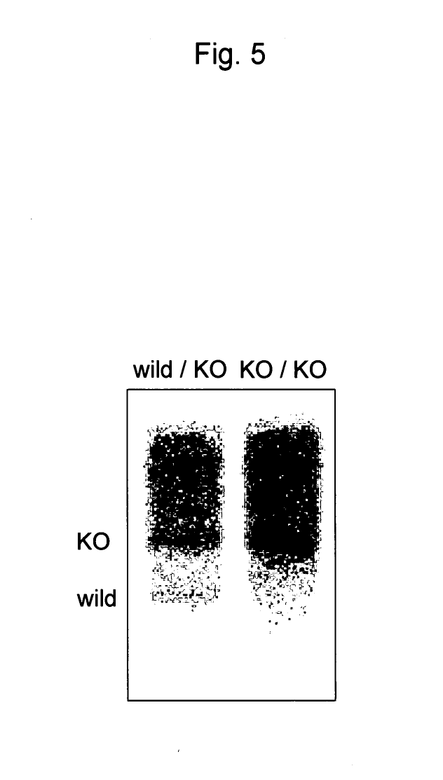

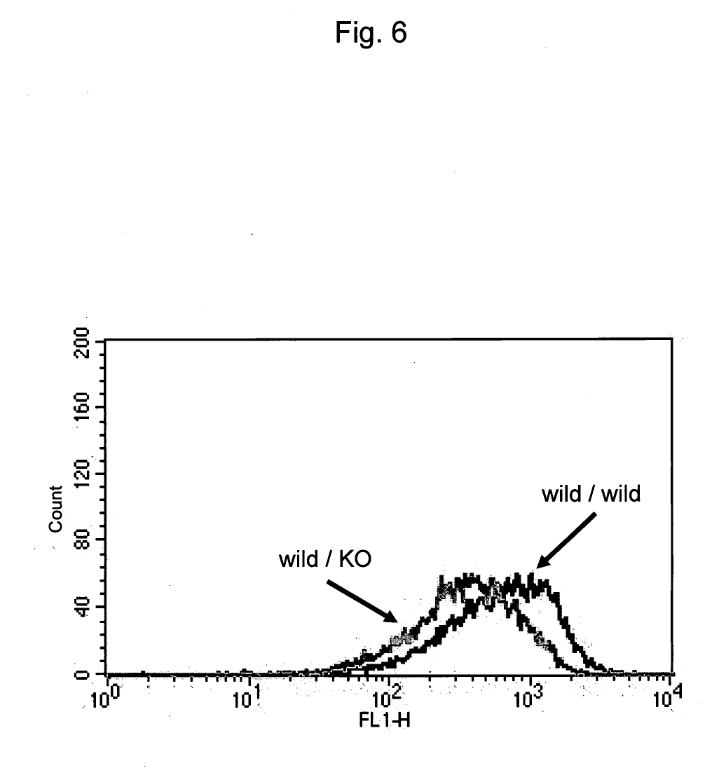

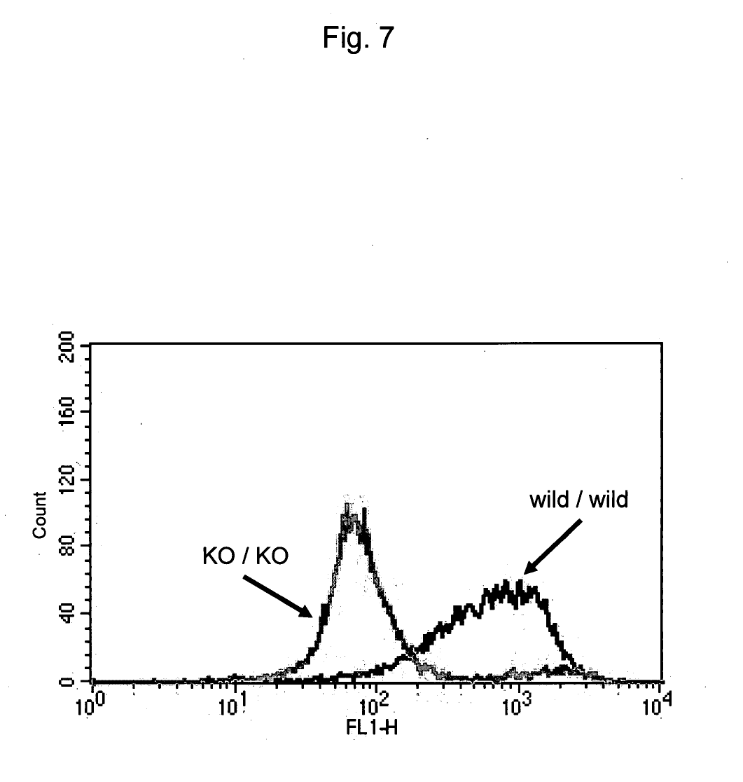

Furthermore, fucose expression in 26 cells that were determined as homologous recombinants by PCR was analyzed. 1×10⁶ cells were stained with 100 μl of PBS (hereinafter, referred to as FACS solution) containing 5 μg/mL Lens culinaris Agglutinin, FITC Conjugate (Vector Laboratories cat. FL-1041), 2.5% FBS, and 0.02% sodium azide for 1 hour during cooling with ice. Subsequently, the cells were washed three times with FACS solution and then analyzed by FACS Calibur (Becton, Dickinson and Company). As a result, it was revealed that only the cells completely lacking the fucose transporter gene as determined by Southern blot analysis exerted decreased fucose expression levels. FIGS. 5 to 7 show the results of Southern blot analysis performed for the obtained clones (FIG. 5) and the results of determining fucose expression levels. FIG. 6 shows the results for wild/KO. FIG. 7 shows the results for KO/KO.

The following was demonstrated by the above results.

Only one cell line completely lacked the fucose transporter gene and the number of such cells screened for was 616. Specifically, homologous recombination frequency was approximately as low as 0.16%. Regarding the knockout of the second step, there are several possible reasons for the inconsistency between PCR analysis results and Southern blot analysis results, as described above. However, in the case of method 3, since selection was performed using 2 types of drug, it was unlikely that the obtained cell lines would contain homologous recombinants by both the KO2 vector alone and the KO3 vector alone. Moreover, it was unlikely that any of the other cell lines determined as homologous recombinants by PCR would be composed of a plurality of cell populations. The presence of 3 or more fucose transporter genes as described above makes targeting in cells significantly difficult. In such case, it was concluded that homologous recombinants may be impossible to obtain unless the KO1 vector, with which Hyg^{r} is weakly expressed, is used and screening would be performed many times.

### Example 2 Construction of Antibody Expression Vectors

A humanized anti-HM1.24 antibody expression vector, AHM(I)/N5KG1 (WO2005/014651), was digested with Ssp I. pCXND3/hGC33/K/Arg that is a vector for expression of an altered antibody gene (reference examples 1 to 3 described later), in which humanized anti-GPC3 antibody H chain is of version K and Asn33 of the L chain had been substituted with Arg, was digested with Pvu I. These vectors were then used for experiments.

### Example 3 Establishment of Antibody-Producing Cells

Hygromycin B was prepared in SFMII (+) medium at a final concentration of 1 mg/ml and the fucose transporter-deficient cell line (FT-KO cell, clone name of 3F2) obtained in Example 1 was maintained. 8×10⁶ 3F2 cells were suspended in 0.8 mL of a Dulbecco's phosphate buffer. 25 μg of each antibody expression vector was added to the cell suspension and then the cell suspension was transferred to a Gene Pulser Cuvette. After the cubette was allowed to stand on ice for 10 minutes, each vector was introduced into the cells by an electroporation method using GENE-PULSER II under conditions of 1.5 kV and 25 μFD. After introduction of the vector, the cells were suspended in 40 mL of SFMII(+) medium and then the suspended cells were inoculated into a 96-well flat bottomed plate (IWAKI) at 100 μl/well. The cells in the plate were cultured within a CO₂ incubator for 24 hours at 37° C., and then Geneticin (Invitrogen Corporation, cat. 10131-027) was added at a final concentration of 0.5 mg/mL. The amounts of antibodies produced by cells that had become resistant to the drug were determined. Thus, a humanized anti-HM1.24 antibody-producing cell line and a humanized anti-GPC3 antibody-producing cell line were each established.

### Example 4 Antibody Purification

The culture supernatant of each antibody-expressing cell line was collected and then Hitrap rProtein A (Pharmacia CAT# 17-5080-01) was charged with the culture supernatant using a P-1 pump (Pharmacia). The resultant was washed using a binding buffer (20 mM Sodium phosphate (pH 7.0)) and then eluted with an elution buffer (0.1M Glycin-HCl (pH 2.7)). The eluate was immediately neutralized with a neutralizing buffer (1M Tris-HCl (pH 9.0)). Eluted fractions of the antibodies were selected using DC protein assay (BIO-RAD CAT# 500-0111) and then pooled. Each resultant was concentrated to approximately 2 mL using Centriprep-YM10 (Millipore CAT# 4304). Next, the resultants were separated by gel filtration using Superdex 200 26/60 (Pharmacia) that had been equilibrated with 20 mM acetate buffer and 150 mM NaCl (pH6.0). Monomer fraction peaks were collected, concentrated using Centriprep-YM10, subjected to filtration using a MILLEX-GW 0.22 μm Filter Unit (Millipore CAT# SLGV 013SL), and then stored at 4° C. Concentrations of the thus purified antibodies were obtained through measurement of absorbances at 280 nm and the following conversion based on molar extinction coefficients.

### Example 5 In Vitro ADCC Activity of a Humanized Anti-HM1.24 Antibody Produced by FT-KO Cells

### 1. Preparation of a Human PBMC Solution

A peripheral blood sample (heparinized blood sample) was obtained from a healthy subject, diluted two-fold using PBS(−), and then layered on Ficoll-Paque™ PLUS (Amersham Biosciences). The resultant was centrifuged (500×g, 30 minutes, 20° C.) and then the intermediate layer that was a mononuclear cell fraction was isolated. The fraction was washed 3 times and then suspended in 10% FBS/RPMI, thereby preparing a human PBMC solution.

### 2. Preparation of Target Cells

ARH-77 cells (ATCC) cultured in 10% FBS/RPMI1640 medium were removed by pipetting from the dish, centrifuged within a tube, and then suspended in 100 μL of medium. 5.55 MBq of Cr-51 was added to the medium and then cultured in a 5% carbon dioxide gas incubator at 37° C. for 1 hour. The cells were washed twice with medium and then 10% FBS/RPMI1640 medium was added to the washed cells. The cells were dispensed into a 96-well U-bottomed plate (Falcon) at 1×04 cells/well, thereby preparing target cells.

### 3. Chromium Release Test (ADCC Activity)

50 μL of an antibody solution prepared at various concentrations was added to the target cells. After 15 minutes of reaction at room temperature, 100 μL of a human PBMC solution was added (5×10⁵ cells/well). The cells were cultured in a 5% carbon dioxide gas incubator at 37° C. for 4 hours. After culture, the plates were subjected to centrifugation and then radioactivity in 100 μL of each culture supernatant was measured using a gamma counter. A specific chromium release percentage was found via the following formula.

Specific chromium release percentage (%)=(A−C)×100/(B−C)

“A” indicates the mean radioactivity (cpm) in each well, “B” indicates the mean radioactivity (cpm) in a well wherein 100 μL of a 2% NP-40 aqueous solution (Nonidet P-40, Code No. 252-23, NACALAI TESQUE, INC.) and 50 μL of 10% FBS/RPMI medium were added to target cells, and “C” indicates the mean radioactivity (cpm) in a well wherein 150 μL of 10% FBS/RPMI medium was added to target cells. The test was performed in tripricate and then means and standard deviations were calculated for ADCC activity (%).

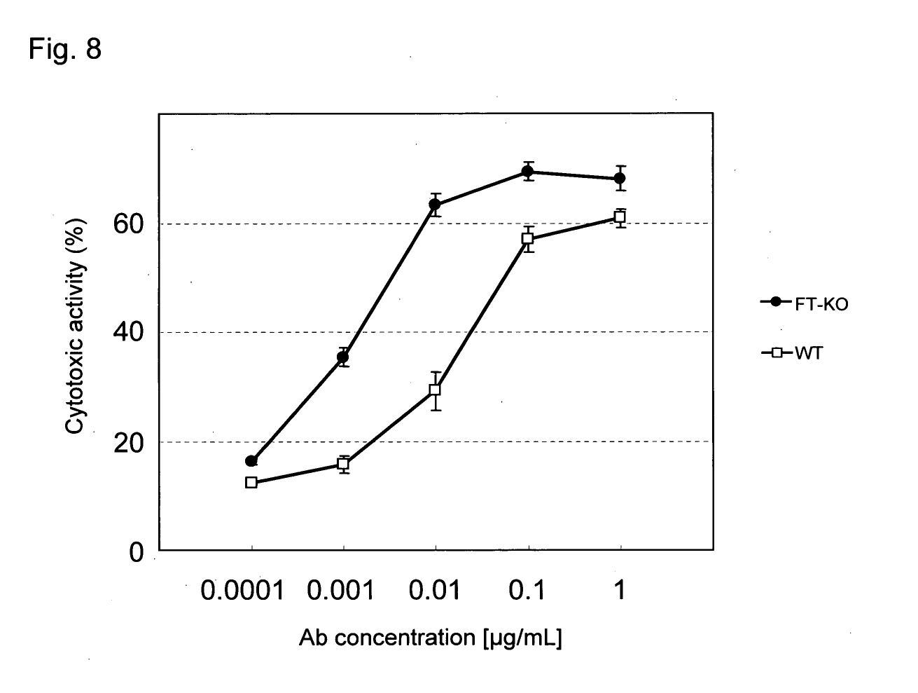

FIG. 8 shows the ADCC activity of the anti-HM1.24 antibodies determined using human PBMC. Open squares indicate the activity of the humanized anti-HM1.24 antibody produced by wild type CHO cells and closed circles indicate the activity of the humanized anti-HM1.24 antibody produced by FT-KO cells. The humanized anti-HM1.24 antibody produced by FT-KO cells exerted stronger ADCC activity than that of the humanized anti-HM1.24 antibody produced by the wild type CHO cells. Thus, it was revealed that the ADCC activity of such antibody produced by FT-KO cells is enhanced.

### Example 6 In Vitro ADCC Activity of Humanized Anti-GPC3 Antibody Produced by FT-KO Cells

### 1. Preparation of Human PBMC Solution

A peripheral blood sample (heparinized blood sample) was obtained from a healthy subject, diluted 2-fold with PBS(−), and then layered on Ficoll-Paque™ PLUS (Amersham Biosciences). The resultant was centrifuged (500×g, 30 minutes, 20° C.) and then an intermediate layer that was a mononuclear cell fraction was isolated. After 3 times of washing, the resultant was suspended in 10% FBS/RPMI, thereby preparing a human PBMC solution.

### 2. Preparation of Target Cells

HuH-7 cells (Health Science Research Resources Bank) cultured in 10% FBS/D-MEM medium were removed from a dish using a Cell Dissociation Buffer (Invitrogen Corporation). The cells were dispensed into a 96-well U-bottomed plate (Falcon) at 1×10⁴ cells/well and then cultured for 1 day. After culture, 5.55 MBq of Cr-51 was added to the cells, followed by 1 hour of culture in a 5% carbon dioxide gas incubator at 37° C. The cells were washed once with medium and then 50 μL of 10% FBS/D-MEM medium was added to the cells, thereby preparing target cells.

### 3. Chromium Release Test (ADCC Activity)

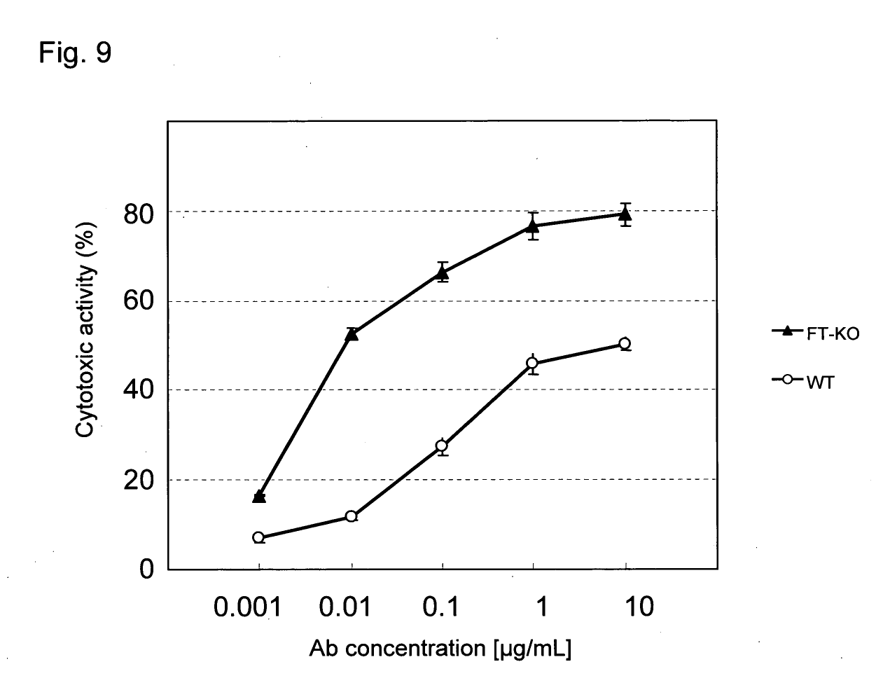

Preparation of a Human PBMC Solution and a Chromium Release Test were performed according to the methods described in Example 1. FIG. 9 shows the ADCC activity of humanized anti-GPC3 antibodies determined using human PBMC. Open circles indicate the activity of the humanized anti-GPC3 antibody produced by wild type CHO cells and closed triangles indicate the activity of the humanized anti-GPC3 antibody produced by FT-KO cells. The humanized anti-GPC3 antibody produced by FT-KO cells exerted stronger ADCC activity than that of the humanized anti-GPC3 antibody produced by wild type CHO cells. Thus, it was revealed that the ADCC activity of such antibody produced by FT-KO cells is enhanced.

### Example 7 Analysis of a Humanized Anti-HM1.24 Antibody Sugar Chain Produced by FT-KO Cells

### 1. Preparation of 2-Aminobenzamide-Labeled Sugar Chains (2-AB-Labeled Sugar Chains)

N-Glycosidase F (Roche diagnostics) was caused to act on the antibody of the present invention produced by FT-KO cells and an antibody produced by CHO cells as a control sample. Sugar chains were then liberated from proteins (Weitzhandler M. et al., Journal of Pharmaceutical Sciences 83: 12 (1994), 1670-1675). After removal of proteins using ethanol (Schenk B. et al., The Journal of Clinical Investigation 108: 11 (2001), 1687-1695), the resultants were concentrated to dryness and then fluorescence-labeled using 2-aminopyridine (Bigge J. C. et al., Analytical Biochemistry 230: 2 (1995), 229-238). The thus obtained 2-AB-labeled sugar chains were subjected to solid phase extraction using a cellulose cartridge for removing the reagent. The resultants were then centrifuged and concentrated, thereby purifying the 2-AB-labeled sugar chains.

### 2. Analysis of Purified 2-AB-Labeled Sugar Chains by Normal Phase HPLC

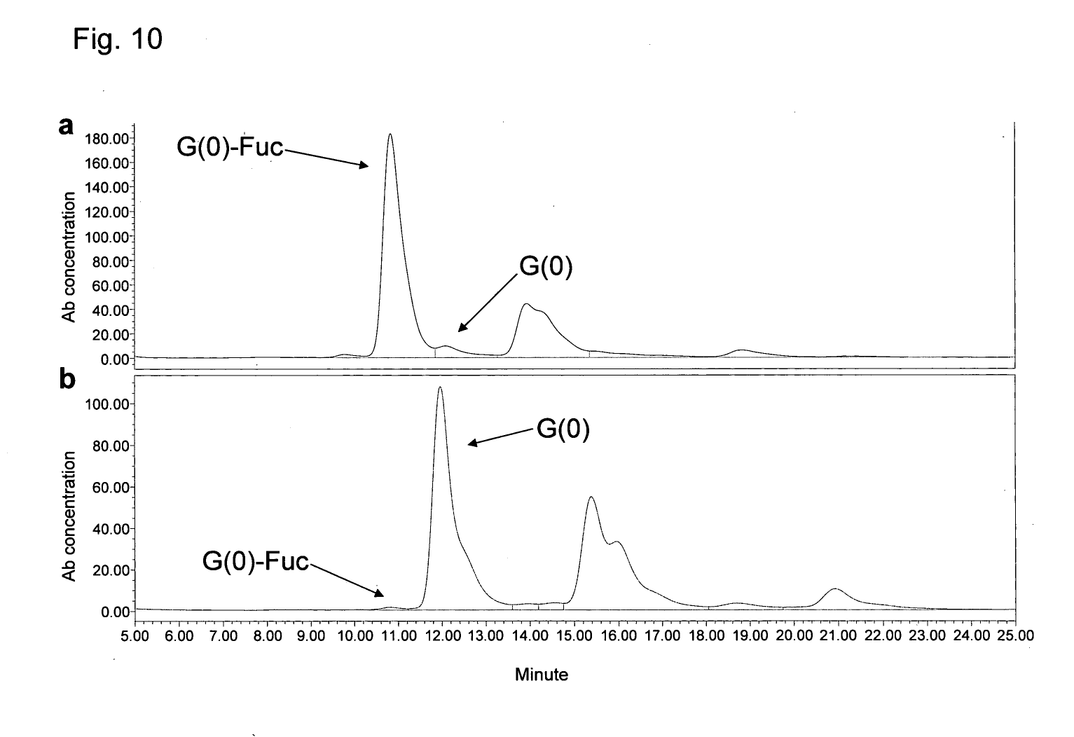

With the method in the previous section, 2-AB-labeled sugar chains were prepared using the antibody of the present invention produced by FT-KO cells and the antibody produced by CHO cells as a control sample. Normal phase HPLC analysis was performed using an amide column (TSKgel Amide-80 produced by TOSOH Corporation) and then the resulting chromatograms were compared. The antibody produced by CHO cells contained G(0-Fuc in a small amount. The proportion of G(0)-Fuc in an agalactosyl sugar chain (G(0)+G(0)-Fuc) was approximately 1.5%. On the other hand, G(0)-Fuc contained in the antibody produced by FT-KO cells accounted for 90% or more (FIG. 10 and Table 1).

### Table 1

Relative Percentage of Each Sugar Chain Inferred from Analysis of Agalactosyl 2-AB-Labeled Sugar Chain by Normal Phase HPLC

| Sugar chain | | |
|---|---|---|
| name | CHO | FT-KO |
| G(0)-Fuc | 1.5% | 92.4% |
| G(0) | 98.5% | 7.6% |

### Example 8 Analysis of Humanized Anti-GPC3 Antibody Sugar Chains Produced by FT-KO Cells

### 1. Preparation of 2-Aminobenzamide-Labeled Sugar Chains (2-AB-Labeled Sugar Chains)

N-Glycosidase F (Roche Diagnostics) was caused to act on the antibodies of the present invention produced by FT-KO cells and an antibody produced by CHO cells as a control sample. Sugar chains were liberated from proteins (Weitzhandler M. et al., Journal of Pharmaceutical Sciences 83: 12 (1994), 1670-1675). After removal of proteins using ethanol (Schenk B. et al., The Journal of Clinical Investigation 108: 11 (2001), 1687-1695), the resultants were concentrated to dryness and then fluorescence labeled with 2-aminopyridine (Bigge J. C. et al., Analytical Biochemistry 230: 2 (1995), 229-238). The thus obtained 2-AB-labeled sugar chains were subjected to solid phase extraction using a cellulose cartridge for removing the reagent. The resultants were then centrifuged and concentrated, thereby purifying the 2-AB-labeled sugar chains. Next, β-Galactosidase (SEIKAGAKU Corporation) was caused to act on the purified 2-AB-labeled sugar chains, thereby preparing agalactosyl 2-AB-labeled sugar chains.

### 2. Analysis of Agalactosyl 2-AB-Labeled Sugar Chains by Normal Phase HPLC

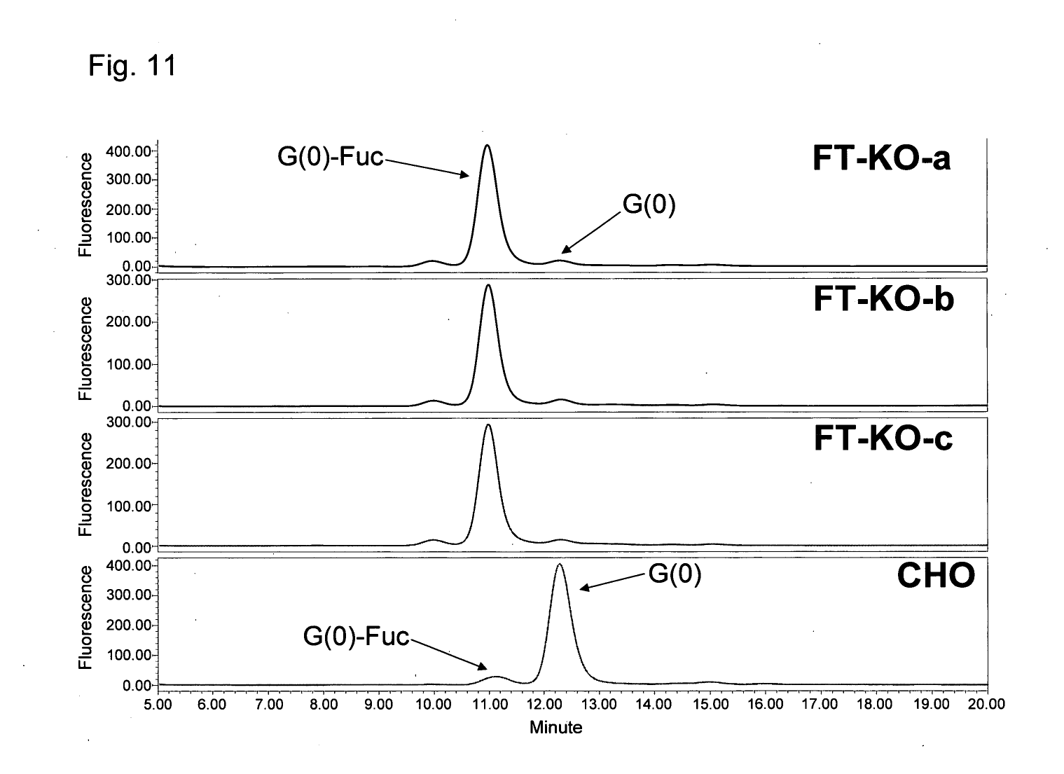

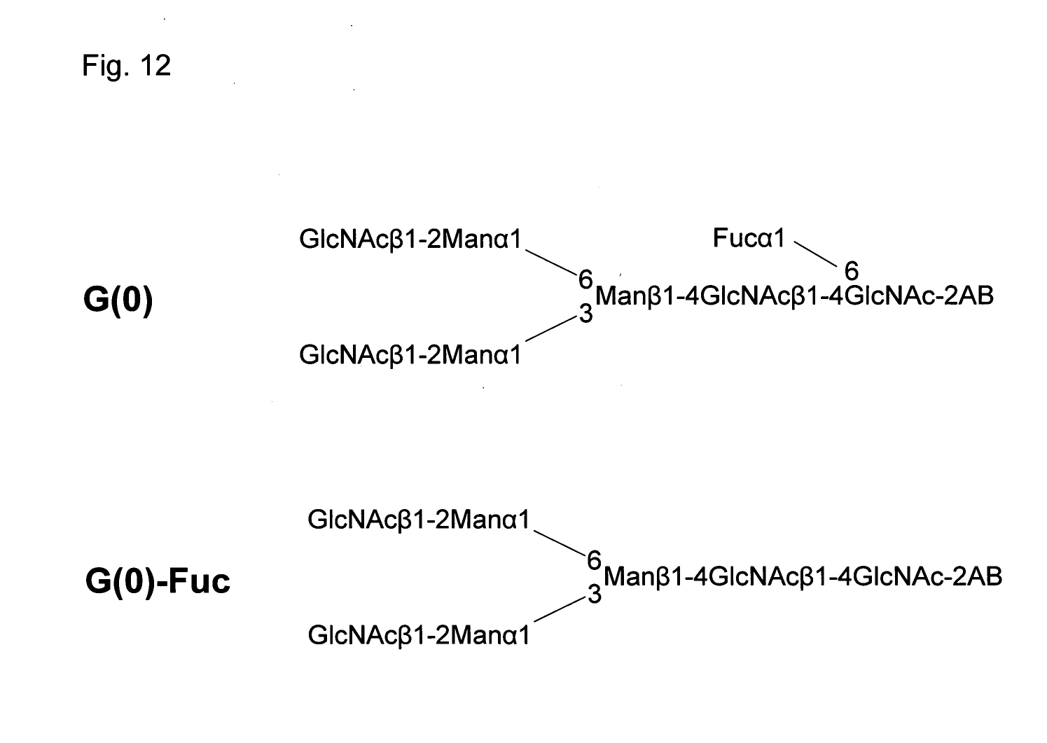

Agalactosyl 2-AB-labeled sugar chains were prepared by the method described in the above section using the antibodies of the present invention produced by FT-KO cells and the antibody produced by CHO cells as a control sample. Normal phase HPLC analysis was performed using an amide column (produced by TOSOH Corporation: TSKgel Amide-80) and then the resulting chromatograms were compared. In the case of the antibody produced by CHO cells, G(0) was contained as a main component; and the peak area proportion of G(0)-Fuc that had not experienced addition of fucose thereto was approximately 4%. On the other hand, in the case of the antibodies produced by FT-KO cells, G(0)-Fuc was contained as a main component; and the peak area proportion of G(0)-Fuc was 90% or more for all the FT-KO cell lines (FIG. 11 and Table 2). FIG. 12 shows the inferred structures of peak G(0) and G(0)-Fuc.

TABLE 2
Relative percentage of each sugar chain inferred from analysis of
agalactosyl 2-AB-labeled sugar chain by normal phase HPLC

| Sugar chain | | | | |
|---|---|---|---|---|
| name | CHO | FT-KO-a | FT-KO-b | FT-KO-c |
| G(0)-Fuc | 4.0% | 92.4% | 92.5% | 93.2% |
| G(0) | 96.0% | 7.6% | 7.5% | 6.8% |

### Example 9 Analysis of Thermostability of a Humanized Anti-GPC3 Antibody Produced by FT-KO Cells

### 1. Preparation of Sample Solutions for DSC Measurement

A 20 mol/L sodium acetate buffer solution (pH 6.0) containing 200 mmol/L sodium chloride was used as a dialysis external solution. A dialysis membrane containing an antibody solution sealed therein in an amount equivalent to 700 μg was immersed in the solution for 24 hours for dialysis, thereby preparing a sample solution.

### 2. Thermal Denaturation Temperature Measurement by DSC

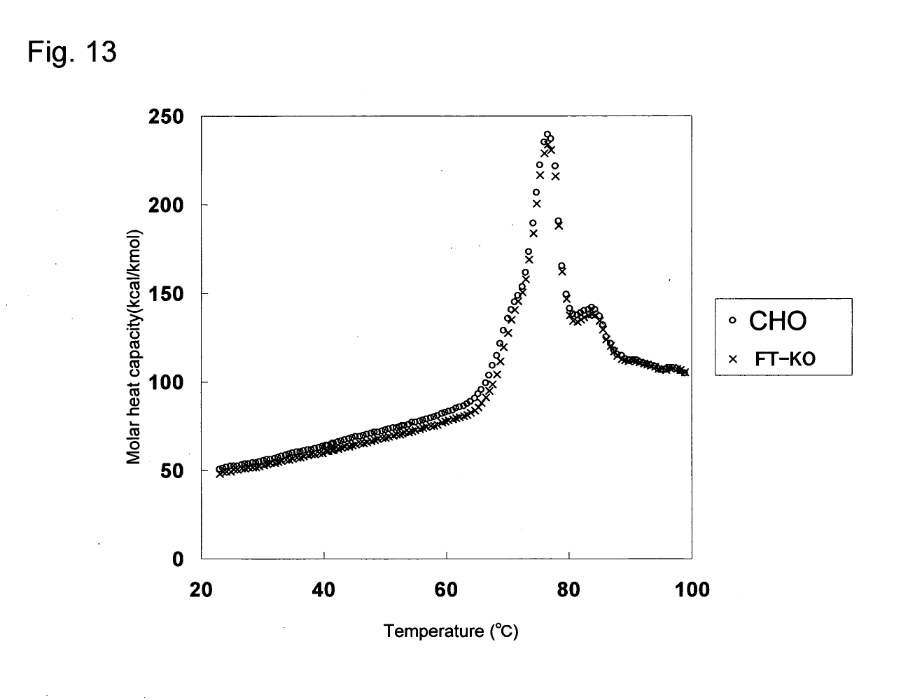

The sample solution and a reference solution (dialysis external solution) were sufficiently deaerated. Each of these solutions was then sealed in a calorimeter cell, followed by sufficient thermal equilibration at 20° C. Next, DSC scanning was performed at a temperature between 20° C. and 100C and a scanning speed of approximately 1K/minute. The resulting denaturation peaks are shown in the form of temperature functions (FIG. 13). In this measurement, the antibody produced by CHO cells and the antibody produced by FT-KO cells were equivalent to each other in terms of thermal denaturation temperature.

### Reference Example 1 Production of Anti-GPC3 Antibody

### 1. Human Glypican 3 (GPC3) cDNA Cloning

A full-length cDNA encoding human GPC3 was amplified by a PCR reaction using 1st strand cDNA as a template prepared from a colon cancer cell line Caco2 by a standard method and an Advantage2 kit (CLONTECH Laboratories, Inc.). Specifically, 50 μl of a reaction solution containing 2 μl of Caco2-derived cDNA, 1 μl of a sense primer (GATATC-ATGGCCGGGACCGTGCGCACCGCGT:SEQ ID NO: 15), 1 μl of an antisense primer (GCTAGC-TCAGTGCACCAGGAAGAAGAAGCAC:SEQ ID NO: 16), 5 μl of an Advantage2 10×PCR buffer, 8 μl of dNTP mix (1.25 mM), and 1.0 μl of Advantage polymerase Mix was subjected to 35 cycles of the PCR reaction, each cycle consisting of 94° C. for 1 minute, 63° C. for 30 seconds, and 68° C. for 3 minutes. An amplified product obtained by the PCR reaction was inserted into TA vector pGEM-T Easy using pGEM-T Easy Vector System I (Promega Corporation). The sequence was confirmed using an ABI3100 DNA sequencer and then cDNA encoding full-length human GPC3 was isolated. The sequence represented by SEQ ID NO: 17 indicates the nucleotide sequence of the human GPC3 gene. The sequence represented by SEQ ID NO: 18 indicates the amino acid sequence of the human GPC3 protein.

### 2. Production of Soluble GPC3

As an immune protein for the production of anti-GPC3 antibodies, a soluble GPC3 protein lacking a C-terminal hydrophobic region (564-580 amino acids) was produced.

PCR was performed using the full-length human GPC3 cDNA as a template, an antisense primer (ATA GAA TTC CAC CAT GGC CGG GAC CGT GCG C: SEQ ID NO: 19) and a sense primer (ATA GGA TCC CTT CAG CGG GGA ATG AAC GTT C: SEQ ID NO: 20) to which an EcoR I recognition sequence and a Kozak sequence had been added. The thus obtained PCR fragment (1711 bp) was cloned into pCXND2-Flag. pCXND2-Flag was designed so that a DHFR gene expression site of pCHOI (Hirata et al., FEBS letter 1994; 356; 244-248) was inserted into the Hind III site in pCXN2 (Niwa et al., Gene 1991; 108; 193-199) and so that a Flag tag sequence was added downstream of the multicloning site for expression of a Flag-tag-added protein. The thus prepared expression plasmid DNA was transfected into a CHO cell line DXB11. Through selection with 500 μg/mL Geneticin, a CHO cell line expressing soluble GPC3 at high levels was obtained. Large-scale culture of the CHO cell line expressing soluble GPC3 at high levels was performed using a 1700-cm² roller bottle. The culture supernatant was collected and then purified. DEAE sepharose Fast Flow (Amersham Biosciences) was charged with the culture supernatant. After washing, elution was performed using a buffer containing 500 mM NaCl. Next, affinity purification was performed using Anti-Flag M2 agarose affinity gel (SIGMA). Elution was performed using 200 μg/mL FLAG peptide. After concentration using Centriprep-10 (Millipore corporation), gel filtration was performed using Superdex 200 HR 10/30 (Amersham Biosciences), so as to eliminate the FLAG peptide. Finally, the resultant was concentrated using a DEAE sepharose Fast Flow column and at the same time elution was performed using PBS (containing 500 mM NaCl) not containing Tween20, thereby performing buffer substitution.

### 3. Preparation of a Soluble GPC3 Core Protein

GPC3 forms a macromolecule as a result of modification by heparan sulfate. To eliminate antibodies against heparan sulfate upon screening for anti-GPC3 antibodies, a soluble GPC3 core protein in which point mutation had been introduced at a heparan sulfate addition site was prepared and then used for screening.

cDNA in which Ser at position 495 and Ser at position 509 had been substituted with Ala was prepared by an assembly PCR method using the above soluble GPC3 (1-563) as a template. At this time, a primer was designed so that an His tag was added to the C-terminus. The thus obtained cDNA was cloned into a pCXND3 vector. pCXND3 was constructed by inserting the DHFR gene expression site in pCHOI into the Hind III site in pCXN2. The thus prepared expression plasmid DNA was transfected into a DXB11 cell line. Through selection using 500 μg/mL Geneticin, a CHO cell line expressing the soluble GPC3 core protein at a high level was obtained.

Large-scale culture was performed using a 1700-cm² roller bottle. The culture supernatant was collected and then purified. Q sepharose Fast Flow (Amersham Biosciences) was charged with the culture supernatant. After washing, elution was performed using phosphate buffer containing 500 mM NaCl. Next, affinity purification was performed using Chelating sepharose Fast Flow (Amersham Biosciences). Gradient elution was performed using 10 mM to 150 mM imidazole. Finally, the resultant was concentrated using Q sepharose Fast Flow and then eluted using a phosphate buffer containing 500 mM NaCl.

As a result of SDS polyacrylamide gel electrophoresis under reduction conditions, 3 bands (70 kDa, 40 kDa, and 30 kDa) were obtained. Amino acid sequencing was performed using an ABI492 protein sequencer (Applied Biosystems). As a result, a 30-kDa band matched an amino acid sequence starting from position 359 or position 375 of GPC3. Thus, it was revealed that GPC3 had been digested between Arg358 and Ser359 or between Lys374 and Val375 so as to be divided into a 40-kDa N-terminal fragment and a 30-kDa C-terminal fragment.

### 4. Preparation of CHO Cells Expressing Full-Length Human GPC3

To obtain a cell line for evaluation of binding activity using flow cytometry, a CHO cell expressing full-length GPC3 was established.

10 μg of full-length human GPC3 gene expression vector was mixed with 60 μL of SuperFect (QIAGEN), thereby forming a complex. The complex was added to the CHO cell line DXB11, so that gene transfer was performed. After 24 hours of culture using a CO₂ incubator, selection was initiated using αMEM (GIBCO BRL) containing Geneticin at a final concentration of 0.5 mg/mL and 10% FBS. The thus obtained Geneticin-resistant colonies were collected, and then cloning of the cells was performed by a limiting dilution method. Each cell clone was solubilized. Full-length human GPC3 expression was confirmed by Western blotting using an anti-GPC3 antibody. Thus, a cell line stably expressing full-length human GPC3 was obtained.

### 5. Evaluation of Binding Activity by ELISA

The soluble GPC3 core protein was diluted to 1 μg/mL using a coating buffer (0.1 mol/L NaHCO₃ (pH 9.6), 0.02% (w/v) NaN₃). The diluted protein was added to an immunoplate and then allowed to stand at 4° C. overnight for coating. Blocking treatment was performed using a dilution buffer (50 mmol/L Tris-HCl (pH 8.1), 1 mmol/L MgCl₂, 150 mmol/L NaCl, 0.05% (v/v) Tween 20, 0.02% (w/v) NaN₃, and 1% (w/v) BSA). An anti-GPC3 antibody was added and then the resultant was allowed to stand at room temperature for 1 hour. After washing with a rinse buffer (0.05% (v/v) Tween 20, PBS), an anti-mouse IgG antibody (ZYMED Laboratories) labeled with alkaline phosphatase was added and then the resultant was allowed to stand at room temperature for 1 hour. After washing with the rinse buffer, SIGMA104 (SIGMA) diluted to 1 mg/mL in a substrate buffer (50 mmol/L NaHCO₃ (pH 9.8) and 10 mmol/L MgCl₂) was added, followed by 1 hour of color development at room temperature. Absorbance (405 nm and reference wavelength of 655 nm) was then measured using a Benchmark Plus (BIO-RAD).

### 6. Immunization with Soluble GPC3 and Selection of Hybridomas

Human GPC3 and mouse GPC3 show homology as high as 94% at the amino acid level. Hence, based on the assumption that anti-GPC3 antibody may be impossible to obtain via immunization of normal mice with GPC3, mice with autoimmune disease, MRL/MpJUmmCrj-lpr/lpr mice (hereinafter, referred to as MRL/lpr mice, purchased from CHARLES RIVER LABORATORIES JAPAN, INC.), were used as animals to be immunized. Immunization was initiated for 7-week-old mice, 8-week-old mice, or older mice. Upon primary immunization, soluble GPC3 prepared at 100 μg/head and then emulsified using Freund's complete adjuvant (FCA, Becton, Dickinson and Company) was subcutaneously administered. 2 weeks later, soluble GPC3 was prepared at 50 μg/head and then emulsified using Freund's incomplete adjuvant (FIA, Becton, Dickinson and Company). The thus emulsified product was subcutaneously administered. Subsequently, booster immunization was performed a total of 5 times at intervals of 1 week. Final immunization was performed by administering soluble GPC3 diluted at 50 μg/head using PBS to 2 of these mice via the tail vein. At 4 days after final immunization, spleen cells were extracted and then mixed with mouse myeloma cells P3-X63Ag8U1 (P3U1, purchased from ATCC) at 2:1. PEG1500 (Roche Diagnostics) was gradually added, so that cell fusion was performed. RPMI1640 medium (GIBCO BRL) was carefully added to dilute PEG1500 and then PEG1500 was removed by centrifugation. The resultant was suspended in RPMI1640 containing 10% FBS and then inoculated at 100 μL/well in a 96-well culture plate. On the next day, RPMI1640 (hereinafter, referred to as HAT medium) containing 10% FBS, 1×HAT media supplement (SIGMA), and 0.5×BM-Condimed H1 Hybridoma cloning supplement (Roche Diagnostics) was added at 100 μL/well. On days 2, 3, and 5, a half of each culture solution was substituted with HAT medium. Screening was performed using culture supernatants on day 7. Screening was performed by ELISA using an immunoplate on which the soluble GPC3 core protein had been immobilized. Positive clones were mono-cloned by a limiting dilution method. As a result, 11 clones (M3C11, M13B3, M1E7, M3B8, M11F1, L9G11, M19B11, M6B1, M18D4, M5B9, and M10D2) of antibodies having strong activity of binding to GPC3 were obtained.

### 7. Cloning of Anti-GPC3 Antibody Variable Regions

Amplification was performed by an RT-PCR method using Total RNA extracted from hybridomas producing anti-GPC3 antibodies. Total RNA was extracted from 1×10⁷ hybridoma cells using RNeasy Plant Mini Kits (QIAGEN). A 5′ terminal gene fragment was amplified using 1 μg of Total RNA, a SMART RACE cDNA Amplification Kit (CLONTECH Laboratories, Inc.), and one of the following synthetic oligonucleotides:

a synthetic oligonucleotide MHC-IgG1 complementary to a mouse IgG1 constant region sequence

| | |
|---|---|
| GGG CCA GTG GAT AGACAG ATG; | (SEQ ID NO: 21) |

a synthetic oligonucleotide MHC-IgG2a complementary to a mouse IgG2a constant region sequence

| | |
|---|---|
| CAG GGG CCA GTG GAT AGA CCG ATG; | (SEQ ID NO: 22) |

a synthetic oligonucleotide MHC-IgG2b complementary to a mouse IgG2b constant region sequence

| | |
|---|---|
| CAG GGG CCA GTG GAT AGA CTG ATG; | (SEQ ID NO: 23) |

and a synthetic oligonucleotide kappa complementary to a mouse K chain constant region nucleotide sequence

| | |
|---|---|
| GCT CAC TGG ATG GTG GGA AGA TG. | (SEQ ID NO: 24) |

A reverse transcription reaction was performed at 42° C. for 1 hour and 30 minutes. 50 μL of a PCR solution contained 5 μL of 10× Advantage 2 PCR Buffer, 5 μL of 10× Universal Primer A Mix, 0.2 mM dNTPs (dATP, dGTP, dCTP, and dTTP), 1 μL of Advantage 2 Polymerase Mix (they were produced by CLONTECH Laboratories Inc.), 2.5 μL of the reverse transcription reaction product, and 10 pmole of synthetic oligonucleotide MHC-IgG1, MHC-IgG2a, MHC-IgG2b, or kappa. A reaction cycle of 94° C. (initial temperature) for 30 seconds, 94° C. for 5 seconds, and 72° C. for 3 minutes was repeated 5 times. A reaction cycle of 94° C. for 5 seconds, 70° C. for 10 seconds, and 72° C. for 3 minutes was repeated 5 times. Furthermore, a reaction cycle of 94° C. for 5 seconds, 68° C. for 10 seconds, and 72° C. for 3 minutes was repeated 25 times. Finally the reaction products were heated at 72° C. for 7 minutes. Each PCR product was purified from agarose gel using a QIAquick Gel Extraction Kit (produced by QIAGEN). The purified product was cloned into a pGEM-T Easy Vector (produced by Promega Corporation) so that each nucleotide sequence was determined.

The nucleotide sequences of the H chain variable regions of M3C11, M13B3, M1E7, M3B8, M11F1, M19B11, M6B1, M18D4, M5B9, M10D2, and L9G11 were represented by SEQ ID NOS: 25, 26, 27, 28, 29, 30, 31, 32, 33, 34, and 35, respectively. The amino acid sequences of the same were represented by SEQ ID NOS: 36, 37, 38, 39, 40, 41, 42, 43, 44, 45, and 46, respectively. The nucleotide sequences of the L chain variable regions of the same were represented by SEQ ID NOS: 47, 48, 49, 50, 51, 52, 53, 54, 55, 56, and 57, respectively. The amino acid sequences of the same were represented by SEQ ID NOS: 58, 59, 60, 61, 62, 63, 64, 65, 66, 67, and 68, respectively.

### 8. Production of Anti-GPC3 Antibody Mouse-Human Chimeric Antibodies

H chain and L chain variable region sequences of anti-GPC3 antibodies were each ligated to human IgG1 and a K chain constant region sequence. PCR was performed using a synthetic oligonucleotide being complementary to the 5′ terminal nucleotide sequence of the H chain variable region of each antibody and having a Kozak sequence and a synthetic oligonucleotide complementary to the 3′ terminal nucleotide sequence having an Nhe I site. The thus obtained PCR product was cloned into a pB-CH vector constructed by inserting the human IgG1 constant region into a pBluescript KS+ vector (TOYOBO Co., Ltd.). Because of the Nhe I site, the mouse H chain variable region had been ligated to the human H chain (γ1 chain) constant region. The thus prepared H chain gene fragment was cloned into an expression vector pCXND3. Furthermore, PCR was performed using a synthetic oligonucleotide being complementary to the 5′ terminal nucleotide sequence of the L chain variable region of each antibody and having a Kozak sequence and a synthetic oligonucleotide complementary to the 3′ terminal nucleotide sequence having a BsiW I site. The thus obtained PCR product was cloned into a pB-CL vector constructed by inserting the human kappa chain constant region into a pBluescript KS+ vector (TOYOBO Co., Ltd.). Because of the BsiW I site, the human L chain variable region had been ligated to the constant region. The prepared L chain gene fragment was cloned into an expression vector pUCAG. The vector pUCAG was constructed by ligating a 2.6-kbp fragment (obtained by digestion of pCXN (Niwa et al., Gene 1991; 108: 193-200) with a restriction enzyme BamH I) to the restriction enzyme BamH I site of a pUC19 vector (TOYOBO Co., Ltd.), followed by cloning.

To construct each anti-GPC3 mouse-human chimeric antibody expression vector, a gene fragment obtained by digestion of the pUCAG vector into which the L chain gene fragment had been inserted with a restriction enzyme Hind III (TAKARA SHUZO CO., LTD.) was ligated to the restriction enzyme Hind III cleavage site of pCXND3 into which an H chain gene had been inserted. Cloning was then performed. This plasmid expresses a neomycin resistance gene, a DHFR gene, and an anti-GPC3 mouse-human chimeric antibody gene in animal cells.

Stable expression cell lines were prepared using CHO cells (DG44 cell line) as follows. Gene transfer was performed by an electroporation method using Gene PulserII (produced by Bio-Rad Laboratories, Inc.). 25 μg of each anti-GPC3 mouse-human chimeric antibody expression vector was mixed with 0.75 ml of CHO cells suspended in PBS (1×10⁷ cells/ml). The mixture was cooled on ice for 10 minutes and then transferred into a cuvette. Pulses were then applied with capacity of 1.5 kV and 25 μFD. After 10 minutes of a recovery period at room temperature, electroporated cells were suspended in 40 mL of CHO—S—SFMII medium (Invitrogen Corporation) containing an HT supplement (Invitrogen Corporation) at the same (1-fold) concentration. A 50-fold diluted solution was prepared using similar medium and then dispensed into a 96-well culture plate at 100 μl/well. After 24 hours of culture in a CO₂ incubator (5% CO₂), Geneticin (Invitrogen Corporation) was added at 0.5 mg/mL, followed by 2 weeks of culture. IgG amounts in the culture supernatants in wells for which colonies of Geneticin-resistant transformed cells had been observed were determined by a concentration determination method as described below. Culture of highly productive cell lines was scaled up in order. Thus, cell lines stably expressing the anti-GPC3 mouse-human chimeric antibodies were obtained and then subjected to large-scale culture, thereby resulting in culture supernatants. Each anti-GPC3 mouse-human chimeric antibody was purified using Hi Trap ProteinG HP (Amersham Biosciences).

### 9. Immunization with GC-3 and Selection of Hybridomas

Of the thus obtained anti-GPC3 antibodies, only M11F1 and M3B8 exerted strong CDC activity. Hence, it was revealed that CDC activity has epitope dependency. For the purpose of obtaining an antibody having both ADCC activity and CDC activity, immunization was performed with GC-3, which is a GST fusion protein containing M11F1 and M3B8 epitopes. GC-3 was purified by the above method in a large amount. The resultant was subjected to gel filtration using Superdex75 (Amersham Biosciences) and then the buffer was substituted with PBS. The product was used as an immune protein. Three Balb/c (purchased from CHARLES RIVER LABORATORIES JAPAN, INC.) and three MRL/lpr mice were immunized with GC-3 according to the above method. Upon primary immunization, GC-3 prepared at 100 μg/head and emulsified using FCA was subcutaneously administered. 2 weeks later, GC-3 prepared at 50 μg/head and then emulsified using FIA was subcutaneously administered. After 5 times of immunization, final immunization (50 μg/head) was performed for all mice through administration via the tail vein so that cell fusion was performed. Screening was performed by ELISA using an immunoplate on which the soluble GPC3 core protein had been immobilized. Positive clones were mono-cloned by a limiting dilution method. As a result, 5 clones (GC199, GC202, GC33, GC179, and GC194) of antibodies having strong activity of binding to GPC3 were obtained.

The H chain and L chain variable regions of GC199, GC202, GC33, GC179, and GC194 were cloned according to the above method and then the sequences thereof were determined. Regarding GC194 L chain, 2 types of sequence were cloned. The nucleotide sequences of the H chain variable regions of GC199, GC202, GC33, GC179, and GC194 were represented by SEQ ID NOS: 69, 70, 71, 72, and 73, respectively. The amino acid sequences of the same were represented by SEQ ID NOS: 74, 75, 76, 77, and 78, respectively. The nucleotide sequences of the L chain variable regions of GC199, GC202, GC33, GC179, GC194(1), and GC194(2) were represented by SEQ ID NOS: 79, 80, 81, 82, 83, and 84, respectively. The amino acid sequences of the same were represented by SEQ ID NOS: 85, 86, 87, 88, 89, and 90, respectively.

### Reference Example 2 Humanization of GC33

Antibody sequence data was obtained from Kabat Database (ftp://ftp.ebi.ac.uk/pub/databases/kabat/) and ImMunoGeneTics Database (IMGT), which are open to the public. The H chain variable regions and the L chain variable regions were separately subjected to homology search. As a result, it was revealed that the H chain variable regions have high homology with DN13 (Smithson et al., Mol. Immunol. 1999; 36: 113-124). It was also revealed that the L chain variable regions have high homology with accession number AB064105, homo sapiens IGK mRNA for immunoglobulin kappa light chain VLJ region, partial cds, clone: K64. Furthermore, the signal sequence of accession Number S40357 having high homology with AB064105 was used for an L chain signal sequence. A complementary determining region (hereinafter, referred to as CDR) was transplanted into the framework region (hereinafter, referred to as FR) of each of these antibodies, so that a humanized antibody was prepared.

Specifically, each synthetic oligo DNA comprising approximately 50 bases was designed, so that approximately 20 bases of such 50-base synthetic oligo DNA undergo hybridization. These synthetic oligo DNAs were assembled by the PCR method, thereby preparing genes each encoding a different variable region. Each of these genes was cloned into an expression vector HEFgγ1 (constructed via cleavage at the Hind III sequence inserted at the end of 5′ terminal synthetic oligo DNA and at the BamH I sequence inserted at the end of 3′ terminal synthetic oligo DNA and cloning of a human IgG1 constant region thereinto) and an expression vector HEFgκ (constructed via cleavage at the same sites and cloning of a human κ chain constant region thereinto) (Sato et al., Mol. Immunol. 1994; 371-381). The H chain and the L chain of the thus prepared humanized GC33 were each named “ver.a.” Humanized GC33 (ver.a/ver.a) in which both H and L chains were “ver.a” exerted binding activity lower than that of an antibody (mouse/mouse) having a mouse GC33 variable region. Through a chimeric combination of a mouse GC33 sequence and ver.a sequence for H chain and L chain, respectively, antibodies (mouse/ver.a and ver.a/mouse) were produced. Then the binding activity of the chimeric antibodies were evaluated. As a result, lowered binding activity was confirmed in ver.a/mouse, revealing that lowered binding activity due to amino acid substitution is caused by an H chain. Hence, altered H chains, ver.c, ver.f, ver.h, ver.i, ver.j, and ver.k, were prepared. All types of humanized GC33 exerted binding activity equivalent to that of chimeric antibodies having the mouse GC33 variable regions. The nucleotide sequences of the humanized GC33H chain variable regions (ver.a, ver.c, ver.f, ver.h, ver.i, ver.j, and ver.k) are represented by SEQ ID NOS: 91, 92, 93, 94, 95, 96, and 97, respectively. The amino acid sequences of the same are represented by SEQ ID NOS: 98, 99, 100, 101, 102, 103, and 104, respectively. The nucleotide sequence of humanized GC33 L chain variable region ver.a is represented by SEQ ID NO: 105 and the amino acid sequence of the same is represented by SEQ ID NO: 106.

In humanized GC33H chain variable regions (ver.i, ver.j, and ver.k), glutamic acid at position 6 had been substituted with glutamine. These antibodies exerted significantly enhanced thermostability.

### Reference Example 3 Alteration of Humanized GC33 L Chain

Regarding protein deamidation, a primary-sequence-dependent deamidation (reaction) rate constant is known. Asn-Gly is known as a sequence that particularly tends to undergo deamidation (Rocinson et al., Proc. Natl. Acad. Sci. U.S.A. 2001; 98; 944-949.). Regarding Asn33 within CDR1 of humanized GC33 L chain ver. a variable region represented by SEQ ID NO: 105, it was predicted that the Asn33 sequence would tend to undergo deamidation since the primary sequence is Asn-Gly.

For evaluation of the effect of Asn33 deamidation on binding activity, an altered antibody was produced in which Asn33 had been substituted with Asp. For introduction of point mutation, a Quick Change Site-Directed Mutagenesis Kit (Stratagene Corporation) was used. Specifically, 50 μL of a reaction solution containing 125 ng of a sense primer (CTT GTA CAC AGT GAC GGA AAC ACC TAT:SEQ ID NO: 107), 125 ng of an antisense primer (ATA GGT GTT TCC GTC ACT GTG TAC AAG: SEQ ID NO: 108), 5 μL of 10× reaction buffer, 1 μL of dNTP mix, 10 ng of HEFgκ in which humanized GC33 L chain ver.a had been cloned, and 1 μL of Pfu Turbo DNA Polymerase was subjected to 12 reaction cycles, each cycle consisting of 95° C. for 30 seconds, 55° C. for 1 minute, and 68° C. for 9 minutes. Subsequently, restriction enzyme Dpn I was added to perform digestion at 37° C. for 2 hours. The resultant was introduced into attached XL1-Blue competent cells, thereby obtaining transformants. Clones in which mutation had been introduced successfully were subjected to excision of variable regions and then cloned into HEFgκ again. The resultants were introduced together with HEFgγ1 in which humanized GC33H chain ver.k had been cloned into COS7 cells using Fugene6 (Roche diagnostics). The culture supernatants of COS7 cells that had been caused to transiently express the antibodies were collected. Antibody concentrations were determined by Sandwich ELISA using an anti-human IgG antibody. The binding activity of the altered antibodies was evaluated by ELISA using an immobilized soluble GPC3 core protein. In the case of the altered antibody (N33D) in which Asn33 had been substituted with Asp, binding activity had disappeared. Hence, it was considered that the effect of Asn33 deamidation on binding activity is significant.

As a method for suppressing Asn33 deamidation, a method that involves altering Gly34 to result in another amino acid has been reported (WO03057881A1). According to this method, altered antibodies G34A, G34D, G34E, G34F, G34H, G34N, G34P, G34Q, G341, G34K, G34L, G34V, G34W, G34Y, G34R, G34S, and G34T, which had experienced substitution with 17 amino acids excluding Cys and Met, were produced using a Quick Change Site-Directed Mutagenesis Kit. Binding activity was evaluated using the culture supernatants of COS7 cells that had transiently expressed the antibodies. As a result, it was revealed that amino acid substitution with those other than Pro(G34P) and Val(G34V) did not affect the maintenance of binding activity.

The amino acid sequences of the light chain CDR1 of the above altered antibodies are shown in SEQ ID NO: 109 (G34A), SEQ ID NO: 110 (G34D), SEQ ID NO: 111 (G34E), SEQ ID NO: 112 (G34F), SEQ ID NO: 113 (G34H), SEQ ID NO: 114 (G34N), SEQ ID NO: 115 (G34T), SEQ ID NO: 116 (G34Q), SEQ ID NO: 117 (G341), SEQ ID NO: 118 (G34K), SEQ ID NO: 119 (G34L), SEQ ID NO: 120 (G34S), SEQ ID NO: 121 (G34W), SEQ ID NO: 122 (G34Y), SEQ ID NO: 123 (G34R), SEQ ID NO: 124 (G34V), and SEQ ID NO: 125 (G34P), respectively. Furthermore, the amino acid sequences of the light chain variable regions of the above altered antibodies are shown in SEQ ID NO: 126 (G34A), SEQ ID NO: 127 (G34D), SEQ ID NO: 128 (G34E), SEQ ID NO: 129 (G34F), SEQ ID NO: 130 (G34H), SEQ ID NO: 131 (G34N), SEQ ID NO: 132 (G34T), SEQ ID NO: 133 (G34Q), SEQ ID NO: 134 (G341), SEQ ID NO: 135 (G34K), SEQ ID NO: 136 (G34L), SEQ ID NO: 137 (G34S), SEQ ID NO: 138 (G34W), SEQ ID NO: 139 (G34Y), SEQ ID NO: 140 (G34R), SEQ ID NO: 141 (G34V), and SEQ ID NO: 142 (G34P), respectively.

### INDUSTRIAL APPLICABILITY

The present invention provides cells in which the expression of fucose transporter genes on both chromosomes is artificially suppressed. When the cells are used for producing a protein, a recombinant protein not having fucose can be produced. Such a recombinant protein not having fucose possesses reduced cytotoxic activity compared with that of proteins having fucose. Furthermore, stability of such a recombinant protein is equivalent to that of proteins having fucose. The protein of the present invention is particularly advantageous when it is used for a recombinant antibody to be used as an antibody drug.

All publications, patents, and patent applications cited herein are incorporated herein in their entirety. A person skilled in the art would easily understand that various modifications and changes of the present invention are feasible within the range of the technical ideas and the scope of the invention as disclosed in the attached claims. The present invention is intended to include such modifications and changes.

1. A cell, in which the expression of fucose transporter genes on both chromosomes is artificially suppressed.

2. The cell according to claim 1, in which the fucose transporter genes are disrupted.

3. The cell according to claim 1 or 2, which is an animal cell.

4. The cell according to claim 3, in which the animal cell is a Chinese hamster cell.

5. The cell according to claim 4, in which the animal cell is a CHO cell.

6. The cell according to any one of claims 2 to 5, in which gene disruption is carried out by homologous recombination using a gene targeting vector.

7. The cell according to claim 1, in which a gene encoding a foreign protein is introduced.

8. The cell according to claim 7, in which the gene encoding the foreign protein is a gene encoding an antibody.

9. A method of producing a protein, comprising culturing the cell according to claim 1.

10. The production method according to claim 9, in which the protein is an antibody.

11. The production method according to claim 10, comprising producing a protein to which fucose is not added.

12. A method for inhibiting the addition of fucose to a protein, comprising artificially suppressing the expression of fucose transporter genes on both chromosomes upon production of a recombinant protein using a cell.

13. The method for inhibiting the addition of fucose to a protein according to claim 12, comprising artificially suppressing gene expression through deletion of a gene.

14. The method for inhibiting the addition of fucose to a protein according to claim 12 or 13, in which the protein is an antibody.

15. The method for inhibiting the addition of fucose to a protein according to claim 12, in which the cell is a CHO cell.
